(19) United States
(12) Patent Application Publication
(10) Pub. No.: US 2025/0171498 A1
(43) Pub. Date: May 29, 2025

(54) ANTIBIOTIC COMPOUNDS, FORMULATIONS AND METHODS OF USE

(71) Applicant: UNIVERSITEIT LEIDEN, Leiden (NL)

(72) Inventors: Nathaniel I. MARTIN, Rapenburg (NL); Jaco SLINGERLAND, Rapenburg (NL)

(73) Assignee: UNIVERSITEIT LEIDEN, Leiden (NL)

(21) Appl. No.: 18/836,507

(22) PCT Filed: Feb. 13, 2023
(86) PCT No.: PCT/NL2023/050065

§ 371 Date: Aug. 7, 2024

(30) Foreign Application Priority Data
Feb. 11, 2022 (NL) 2030897

(51) Int. Cl. C07K 7/62; A61K 38/12; A61P 31/04

(52) U.S. Cl. CPC C07K 7/62; A61K 38/12; A61P 31/04

(57) ABSTRACT

Antibiotic compounds Provided herein are modified polymyxin compounds of formula (I): (I), wherein: R^{A}; R^{B}; R^{C}; R^{D}; R^{E}; and R^{F} each individually represents hydrogen, branched or linear chain C₁-C₄ alkyl, optionally substituted with a hydroxyl suflhydryl, alkyl thiol ether; β-carboxyl or γ-carboxyl, aromatic or heteroaromatic substituent; R¹ represents an optionally substituted alkyl moiety, an optionally substituted benzyl moiety; R² represents hydrogen, hydroxymethyl, aminoethyl, aminomethyl, or 5-(2-amino)pentanoic acid; R³ represents —NH₂ or —N(H)—COCH₂NH₂; R⁴ represents an optionally substituted alkyl moiety, an optionally substituted cycloalkyl moiety; or an optionally substituted aryl moiety; R⁵ and R⁶ each independently represent hydrogen or an optionally substituted alkyl moiety; each X independently represents C, S, O, or N; or a pharmaceutically acceptable salt, stereoisomer, solvate, or prodrug thereof. Also provided are compositions comprising such compounds; as well as such compounds or formulations for use as a medicament, for use in the treatment of bacterial infection.

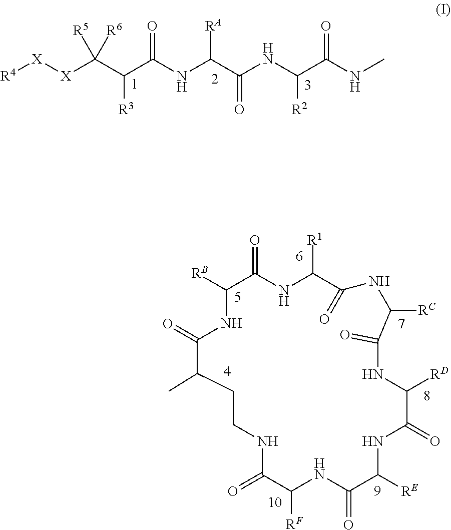

This invention relates to novel antibiotic compounds, especially a novel class of polymyxins. The compounds are active against Gram-negative bacteria. The invention also provides processes for making the novel antibiotic compounds. The invention further relates to formulations comprising the novel antibiotic compounds. Also provided are methods of using such antibiotic compounds and such formulations for treating a bacterial infection.

### BACKGROUND

Worldwide, the emergence of multiple-drug resistant bacteria is on the rise while the pipeline of new antibiotics under development is nearly dry. This problem is most notable when it comes to the dearth of new antibiotics that target Gram-negative species.

Polymyxins are a clinically established class of antibiotics, on the market since the 1960's. They act exclusively on mostly difficult to treat Gram-negative pathogens. As resistance to many other antibiotics continues to rise, and Gram-negative bacteria are inherently difficult to treat with current antibiotics, polymyxins have become a last resort therapy for clinicians. Clinically used polymyxin family members are polymyxin B and its closely related analogue polymyxin E (also known as colistin).

Structurally, polymyxins contain a macrocyclic heptapeptide, ring-closed by the C-terminus of the peptide and the side chain of the 2,4-diaminobutyric acid (Dab) residue found at position 4, along with an exocyclic tripeptide that is acylated at the N-terminus with a fatty acid tail. Polymyxin B and colistin differ only in the amino acid residue found at position 6 which is D-Phe in polymyxin B and D-Leu in colistin. Like other amphiphilic cationic antimicrobial membrane active compounds, the polymyxins selectively target bacterial membranes over mammalian membranes.

Despite their potent antibacterial activity, polymyxins sufferfrom a serious drawback. Their clinical application is dose limited due to their well-documented nephrotoxicity, an effect that has historically limited their widespread use in treating infections, see for instance Akajagbor, D. S. et al.; Higher Incidence of Acute Kidney Injury With Intravenous Colistimethate Sodium Compared With Polymyxin B in Critically Ill Patients at a Tertiary Care Medical Center. Clin. Infect. Dis. 2013, 57 (9), 1300-1303; and P., Z. A.; L., N. R. Nephrotoxicity of Polymyxins: Is There Any Difference between Colistimethate and Polymyxin B?Antimicrob. Agents Chemother. 2017, 61 (3), e02319-16.

Kidney failure is a common reason to halt antibacterial treatment. The toxic effect of polymyxins is largely driven by their tendency to accumulate in kidney tubular cells.

However, with the increasing incidence of MDR Gram-negative pathogens, the use of polymyxin therapy is on the rise, see for instance Evans, M. E. et al.; Polymyxin B Sulfate and Colistin: Old Antibiotics for Emerging Multiresistant Gram-Negative Bacteria. Ann. Pharmacother. 1999, 33 (9), 960-967. Hence, the demand for safer variants is growing.

Two ways to increase the therapeutic window of polymyxins were thus far focusing on either enhancing antibacterial activity, preferably without increasing toxicity, or reducing toxicity, preferably without losing antibacterial activity.

Several approaches have been explored to assess more active or less toxic polymyxin analogues. Some analogues found exhibit low acute toxicity, but the nephrotoxicity appeared to be higher. However, as the molecular basis of polymyxin toxicity is not completely understood, it is hard to find polymyxin analogues that are both effective against Gram-negative bacteria and show an acceptable nephrotoxicity.

An object of the invention is therefore to provide compounds that are useful in the treatment of infections with Gram-negative bacteria. A further object is to provide a novel class of polymyxins that show a toxicity similar to, but preferably less than, Polymyxin B and Colistin, while at the same time show very good antibacterial activity similar to Polymyxin B and Colistin.

Furthermore, a further object the aim is to provide a novel class of polymyxins that can be produced in a cost effective and environmentally friendly way. Lastly, an object is also provision of method to modify polymyxin analogues, preferably Polymyxin B or E analogues, such that their nephrotoxicity is reduced while still acting against bacterial pathogens.

### BRIEF SUMMARY OF THE DISCLOSURE

Accordingly, in a first aspect, the present invention provides compounds that are useful in the treatment of bacterial infection. For example, the compounds may be useful in the treatment of an infection by Gram-negative bacteria. Compounds of the invention are polymyxins backbones comprising a lipid side chain, preferably a disulfide-containing lipid side chain.

Therefore, the invention provides in a first aspect a compound of formula (I):

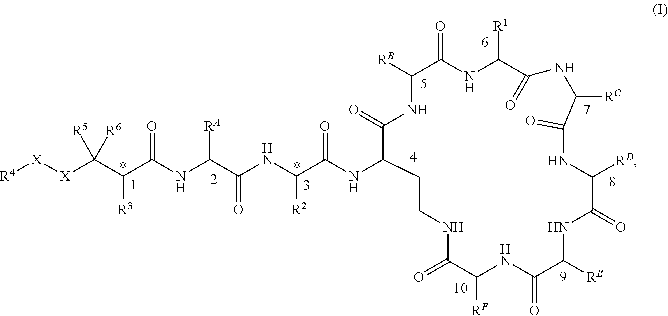

wherein:

R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with hydroxyl, suflhydryl, alkyl thiol ethers, carboxyl, preferably β-carboxyl or γ-carboxyl, aromatic or heteroaromatic substituents, preferably benzyl, guanidinium or imidazolium, and/or amino groups, preferably ε-NH₃⁺;

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety; each X independently represents C, S, O, or N;

or a pharmaceutically acceptable salt, stereoisomer, solvate, or prodrug thereof.

Herein, the bond X—X may represent C—C, C—S, C—O, C—N, S—S, S—C, S—O, S—N, O—O, O—C, O—S, O—N, N—N, N—C, N—S, or N—O, preferably C—C, S—S, C—S, S—C, C—O or O—C, more preferably C—C or S—S, most preferably S—S.

The term “side chain of a natural or non-natural alpha-amino acid” means the group Rx (i.e. R^{A}; R^{B}; R^{C}; R^{D}; R^{E}; and/or R^{F}) in a natural or non-natural amino acid of formula NH₂—CH(R^{x})—COOH, with the proviso that sulfur or oxygen substituents directly connected to the amine acid backbone, e.g. NH₂—CH(OR)—COOH or NH₂—CH(SR)—COOH are not preferred due to the observed reduction in biological activity.

Preferred are compounds of formula (Ia):

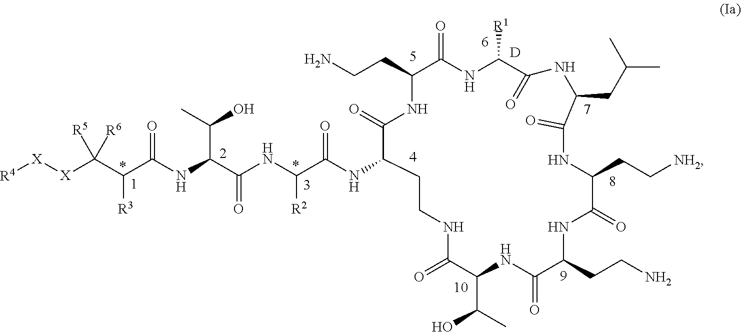

wherein:

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety, preferably methyl; or a pharmaceutically acceptable salt, stereoisomer, solvate, or prodrug thereof.

Preferably, in the compounds of formula (Ia), R¹ represents one of the following structures:

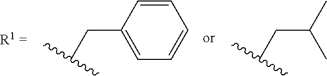

Preferably, in the compounds of formula (Ia), R⁵ and R⁶ each independently represent hydrogen.

A further preferred compound is a compound of formula (II):

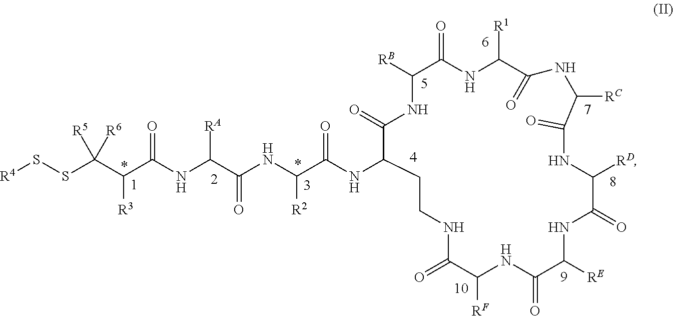

wherein:

R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with hydroxyl, suflhydryl, alkyl thiol ethers, carboxyl, preferably β-carboxyl or γ-carboxyl, aromatic or heteroaromatic substituents, preferably benzyl, guanidinium or imidazolium, and/or amino groups, preferably ε-NH₃⁺;

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety; or a pharmaceutically acceptable salt, stereoisomer, solvate, or prodrug thereof.

Preferred are compounds of formula (IIa):

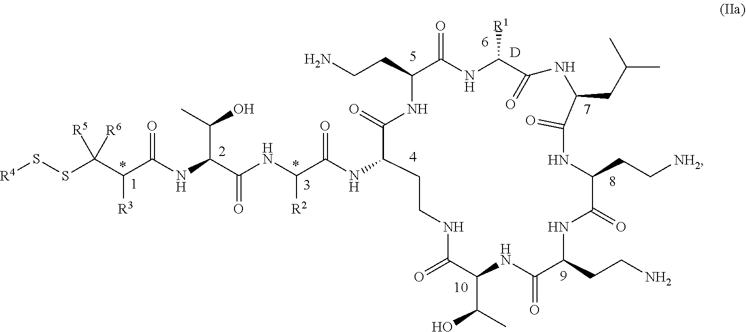

wherein:

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety, preferably methyl; or a pharmaceutically acceptable salt, stereoisomer, solvate, or prodrug thereof.

Preferably, in the compounds of formula (IIa), R¹ represents one of the following structures:

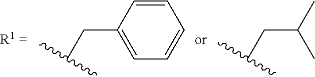

Preferably, in the compounds of formula (IIa), R⁵ and R⁶ each independently represent hydrogen.

Preferred are compounds of formula (IIb):

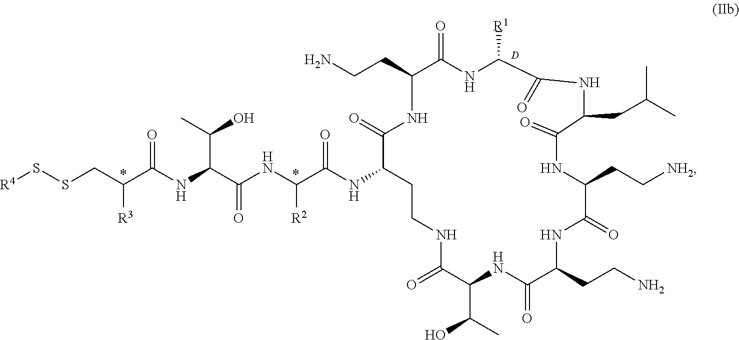

wherein:
R¹ represents an optionally substituted straight or branched chain alkyl, alkenyl, alkinyl or alkylene moiety having up to 20 carbon atoms; optionally substituted with an aryl or heteroaryl moiety;
R² represents hydrogen, hydroxymethyl, 2-aminoethyl, aminomethyl, or 5-(2-amino)pentanoic acid;
R³ represents —NH₂ or —N(H)—COCH₂NH₂; and R⁴ represents an optionally substituted alkyl moiety, an optionally substituted cycloalkyl moiety, or an optionally substituted aryl moiety or an optionally substituted arylalkyl moiety;
or a pharmaceutically acceptable salt, stereoisomer, solvate, or prodrug thereof.

Preferably, in the compounds of formula (IIb), R¹ represents one of the following structures:

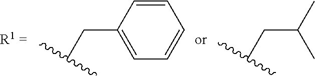

A further preferred compound is a compound of formula (III):

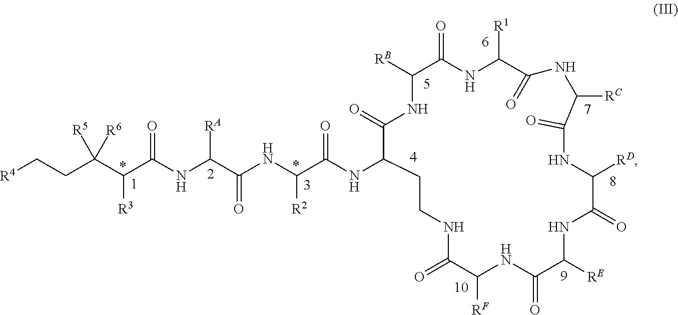

wherein:

R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with hydroxyl, suflhydryl, alkyl thiol ethers, carboxyl, preferably β-carboxyl or γ-carboxyl, aromatic or heteroaromatic substituents, preferably benzyl, guanidinium or imidazolium, and/or amino groups, preferably ε-NH₃⁺;

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety; or a pharmaceutically acceptable salt, stereoisomer, solvate, or prodrug thereof.

Preferred are compounds of formula (IIIa):

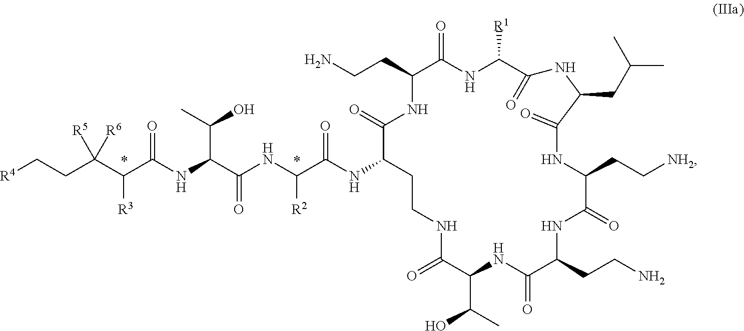

wherein:

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety, preferably methyl; or a pharmaceutically acceptable salt, stereoisomer, solvate, or prodrug thereof.

Preferably, in the compounds of formula (IIIa), R¹ represents one of the following structures:

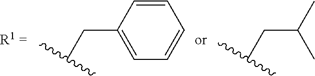

Preferably, in the compounds of formula (IIIa), R⁵ and R⁶ each independently represent hydrogen.

In a second aspect of the invention a compound of formula (IV) is provided:

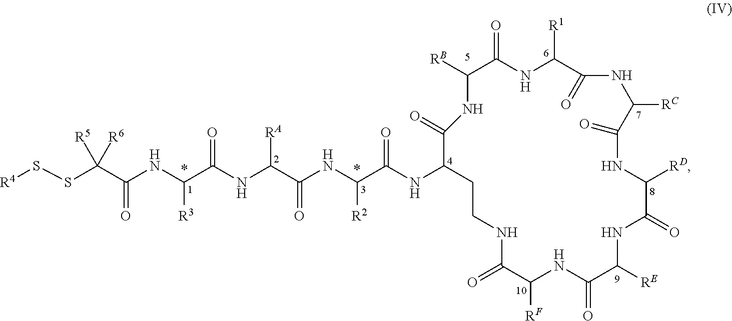

wherein:

R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with hydroxyl, suflhydryl, alkyl thiol ethers, carboxyl, preferably β-carboxyl or γ-carboxyl, aromatic or heteroaromatic substituents, preferably benzyl, guanidinium or imidazolium, and/or amino groups, preferably ε-NH₃⁺;

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety; or a pharmaceutically acceptable salt, stereoisomer, solvate, or prodrug thereof.

Preferred are compounds of formula (IVa):

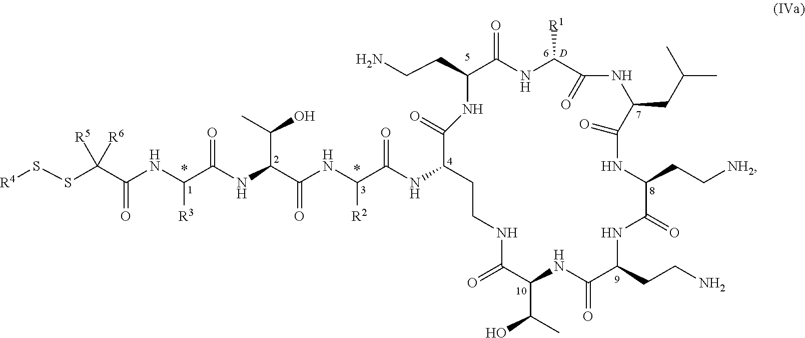

wherein:

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety, preferably methyl; or a pharmaceutically acceptable salt, stereoisomer, solvate, or prodrug thereof.

Preferably, in the compounds of formula (IVa), R¹ represents one of the following structures:

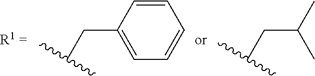

Preferably, in the compounds of formula (IVa), R⁵ and R⁶ each independently represent hydrogen.

In a third aspect of the invention a compound of formula (V) is provided:

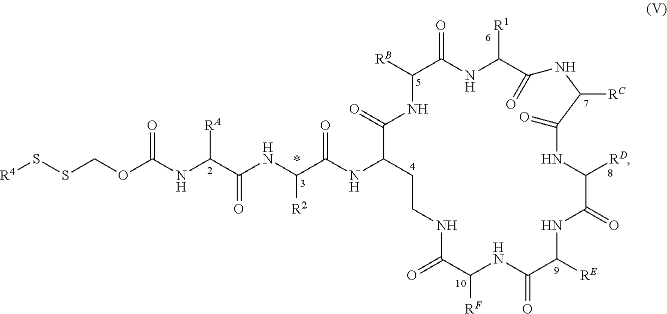

wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}. R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃⁺;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R⁴ represents an optionally substituted alkyl moiety, an optionally substituted cycloalkyl moiety; or an optionally substituted aryl moiety;
or a pharmaceutically acceptable salt, stereoisomer, solvate, or prodrug thereof.

Preferred are compounds of formula (Va):

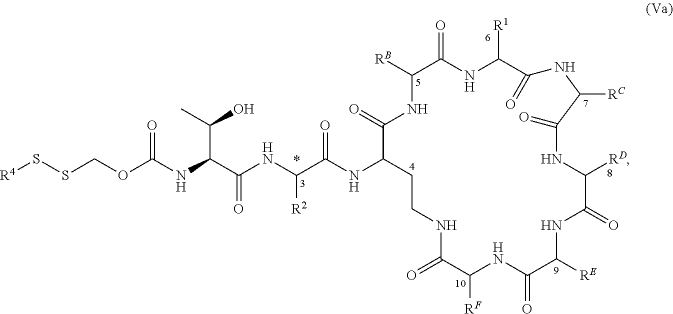

wherein:

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R⁴ represents an optionally substituted alkyl moiety, an optionally substituted cycloalkyl moiety; or an optionally substituted aryl moiety;

or a pharmaceutically acceptable salt, stereoisomer, solvate, or prodrug thereof.

Preferably, in the compounds of formula (Va), R¹ represents one of the following structures:

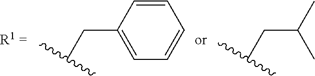

In a fourth aspect of the invention a compound of formula (VI) is provided:

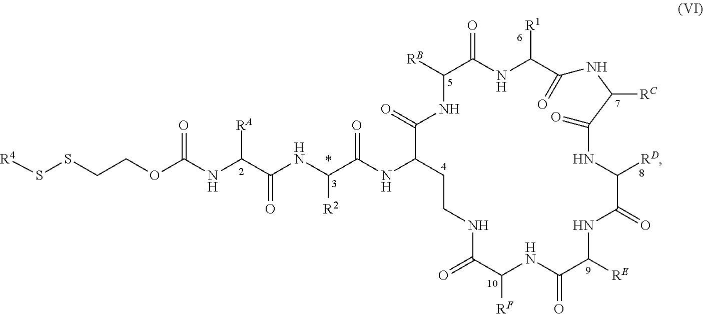

wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}. R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃⁺;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R⁴ represents an optionally substituted alkyl moiety, an optionally substituted cycloalkyl moiety; or an optionally substituted aryl moiety;
or a pharmaceutically acceptable salt, stereoisomer, solvate, or prodrug thereof.

Preferred are compounds of formula (VIa):

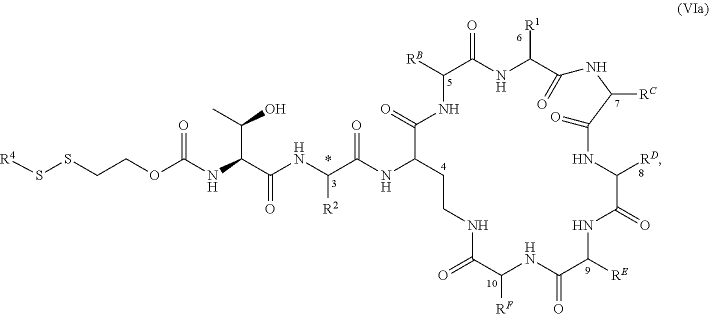

wherein:

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R⁴ represents an optionally substituted alkyl moiety, an optionally substituted cycloalkyl moiety; or an optionally substituted aryl moiety;

or a pharmaceutically acceptable salt, stereoisomer, solvate, or prodrug thereof.

Preferably, in the compounds of formula (VIa), R¹ represents one of the following structures:

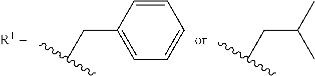

A fifth aspect of the invention provides a process for making the compound of the invention.

A sixth aspect of the invention provides a composition of the invention comprising compound of the invention and a pharmaceutically acceptable carrier. The composition may be a parenteral formulation or an oral formulation. The formulation may be a parenteral formulation, such as a formulation for intravenous injection.

A seventh aspect provides a compound or composition of the invention for use as a medicament.

An eighth aspect provides a compound or composition of the invention for use in the treatment of a bacterial infection. The bacterial infection may be an infection by Gram-negative bacteria. The Gram-positive bacteria may be from at least one of the following families.

A ninth aspect provides a method of treating a bacterial infection in a patient, comprising administering to the patient an effective amount of a compound of the invention, or composition of the invention.

Therefore, in further aspects, the present invention also encompasses one or more processes for the preparation of the compounds; the pharmaceutical use of a compound or composition of the invention; a pharmaceutical composition comprising a compound or a composition of the invention together with a pharmaceutically acceptable diluent or carrier; the use of a compound or composition of the invention in the preparation of a medicament for treating or preventing septic shock; and a method for treating or preventing septic shock, which comprises administering a therapeutically or prophylactically effective amount of a compound or composition of the invention, to an individual in need

A compound or composition according to the invention may advantageously be administered to mammals, preferably humans, when a Gram-negative bacteria infection is diagnosed, e.g. those that may lead to endotoxicosis, bacterial sepsis and/or septic shock.

Gram-negative bacteria that may be responsible for these fatal disorders include, but are not limited to N. meningitidis, E. coli, Salmonella typhi, Bordetella pertussis and Pseudomonas aeruginosa. A compound or composition of the invention may be administered to an individual in need by a systemic route, preferably the intravenous route. The dose to be administered depends on various factors including, but not limited to the age, weight, physiological condition of the patient as well as the infection status. It may be administered once or several times until the risk of fatal event is avoided.

The invention provides a novel class of polymyxins that show a reduced toxicity as compared to Polymyxin B and Colistin, and at the same time show very good antibacterial activity similar to Polymyxin B and Colistin. Furthermore, these polymyxins can be produced a cost effective and environmentally friendly way.

### BRIEF DESCRIPTION OF THE DRAWINGS

Embodiments of the invention are further described hereinafter with reference to the accompanying drawings, in which:

FIG. 1 illustrates Synthesis Scheme 1.

FIG. 2 illustrates Synthesis Scheme 2.

FIG. 3 illustrates Synthesis Scheme 3.

FIG. 4 illustrates Synthesis Scheme 4.

FIG. 5 illustrates Synthesis Scheme 5.

FIG. 6 illustrates Synthesis Scheme 6.

FIG. 7 illustrates Synthesis Scheme 7.

FIG. 8 illustrates Synthesis Scheme 8.

FIG. 9 illustrates Synthesis Scheme 9.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

All publications, patent applications, patents, and other references mentioned herein are incorporated by reference in their entirety. In case of conflict, the present specification, including definitions, will control.

### Definitions

The following explanations of terms and methods are provided to better describe the present disclosure and to guide those of ordinary skill in the art in the practice of the present disclosure.

Gram-negative bacteria that are resistant to aminoglycoside, β-lactam, and fluoroquinolone antibiotics are increasingly common. These bacteria are often only susceptible to the polymyxins and related peptides having antibacterial properties. As a result, there is renewed interest in the use of polymyxins for the treatment of multidrug-resistant Gram-negative bacterial infections in humans.

Peptides such as polymyxin B and the related colistin, also referred to as polymyxin E, have been administered to humans as antibacterial agents. However, their use has been previously limited because of their toxicity. Thus, there is a need for new peptide compounds having equivalent antibacterial properties to polymyxin B with an improved therapeutic index, as well as methods of manufacturing such antibacterial compounds.

The invention concerns amongst other things the treatment of a disease. The term “treatment”, and the therapies encompassed by this invention, include the following and combinations thereof: (1) hindering, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission). The benefit to a patient to be treated may be either statistically significant or at least perceptible to the patient or to the physician. It will be understood that a medicament will not necessarily produce a clinical effect in each patient to whom it is administered; thus, in any individual patient or even in a particular patient population, a treatment may fail or be successful only in part, and the meanings of the terms “treatment” and “prophylaxis” and of cognate terms are to be understood accordingly. The compositions and methods described herein are of use for therapy and/or prophylaxis of the mentioned conditions.

The term “prophylaxis” includes reference to treatment therapies for the purpose of preserving health or inhibiting or delaying the initiation and/or progression of an event, state, disorder or condition, for example for the purpose of reducing the chance of an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

The term “antibiotic” refers to a compound that inhibits the growth of or destroys microorganisms, such as bacteria (e.g. Gram-positive bacteria, or Gram-negative bacteria). An “antibacterial” is an antibiotic that is active against bacteria.

Compounds of the invention are antibacterial, in particular with activity against Gram-negative bacteria. Gram-positive bacteria include Staphylococcus (e.g. S. aureus, S. epidermidis, S. saprophyticus), Streptococcus (e.g. Strep. pyogenes, Strep. agalactiae, Strep. viridans, Strep. pneumonia), Enterococus, Bacillus, Clostridia, Listeria and Corynebacterium.

The term “alkyl” as used herein includes reference to a straight or branched chain alkyl moiety having up to 20 (e.g. 1, 2, 3, 4, 5, 6, 7, 8, 9, 10, 11, 12, 13, 14, 15, 16, 17, 18, 19 or 20) carbon atoms. The term includes reference to, for example, methyl, ethyl, propyl (n-propyl or isopropyl), butyl (n-butyl, sec-butyl or tert-butyl), pentyl, hexyl and the like. In particular, alkyl may be a “C₁-C₁₀ alkyl”, i.e. an alkyl having 1, 2, 3, 4, 5, 6, 7, 8, 9 or 10 carbon atoms; “C₁-C₆ alkyl”, i.e. an alkyl having 1, 2, 3, 4, 5 or 6 carbon atoms; “C₁-C₄ alkyl”, i.e. an alkyl having 1, 2, 3 or 4 carbon atoms; a “C₁-C₆ alkyl”, i.e. an alkyl having 1, 2, 3, 4, 5 or 6 carbon atoms; or a “C₁-C₃ alkyl”, i.e. an alkyl having 1, 2 or 3 carbon atoms. The term “lower alkyl” includes reference to alkyl groups having 1, 2, 3 or 4 carbon atoms.

The term “alkenyl” as used herein includes reference to a straight or branched chain alkenyl moiety having up to 20 (e.g. 2, 3, 4, 5, 6, 7, 8, 9, 10, 11, 12, 13, 14, 15, 16, 17, 18, 19 or 20) carbon atoms. The term includes reference to, for example, ethenyl, propenyl, butenyl, pentenyl, hexenyl and the like. In particular, alkenyl may be a “C₂-C₁₀ alkenyl”, i.e. an alkenyl having 2, 3, 4, 5, 6, 7, 8, 9 or 10 carbon atoms; “C₂-C₆ alkyl”, i.e. an alkenyl having 2, 3, 4, 5 or 6 carbon atoms; “C₂-C₄ alkyl”, i.e. an alkenyl having 1, 2, 3 or 4 carbon atoms; The term “lower alkenyl” includes reference to alkyl groups having 2, 3 or 4 carbon atoms. The alkenyl may be monounsaturated (i.e. comprise a single carbon carbon double bond) or polyunsaturated (i.e. comprise a two or more carbon carbon double bonds, e.g. 2, 3 or 4 carbon carbon double bonds). For example, an alkenyl may be an alkadienyl, alkatrienyl, etc.

The term “alkylene” by itself or as part of another substituent means a divalent radical derived from an alkyl, as exemplified, but not limited, by —CH2CH2CH2CH2-. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “cycloalkyl” as used herein includes reference to an alicyclic moiety having 3, 4, 5 or 6 carbon atoms. The group may be a bridged or polycyclic ring system. More often cycloalkyl groups are monocyclic. This term includes reference to groups such as cyclopropyl, cyclobutyl, cyclopentyl, cyclohexyl and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. For example, a heterocycloalkyl may comprise 3, 4, or 5 ring carbon atoms and 1 or 2 ring heteroatoms selected from nitrogen and oxygen. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

The terms “halo” or “halogen” as used herein includes reference to F, Cl, Br or I, for example F, Cl or Br. In a particular class of embodiments, halogen is F or Cl, of which F is more common.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “haloalkyl” refers to an alkyl group where one or more hydrogen atoms are substituted by a corresponding number of halogens. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms, e.g. 1, 2 or 3 carbon atoms. This term includes reference to, for example, methoxy, ethoxy, propoxy, isopropoxy, butoxy, tert-butoxy, pentoxy, hexoxy and the like. The term “lower alkoxy” includes reference to alkoxy groups having 1, 2, 3 or 4 carbon atoms.

The term “haloalkoxy” as used herein refers to an alkoxy group where one or more hydrogen atoms are substituted by a corresponding number of halogens.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Arylene” and “heteroarylene” refers to a divalent radical derived from an aryl and heteroaryl, respectively.

The term “lipid” with reference to a substituent as used herein represents a moiety that is typically hydrophobic. A lipid may comprise substituted or unsubstituted alkyl, alkenyl, cycloalkyl, bridged cycloalkyl, (alkyl)cycloalkyl, (alkyl) bridged cycloalkyl, (alkyl)cycloalkenyl, and/or alkylaryl groups. For example, a lipid may comprise substituted or unsubstituted alkyl, alkenyl, (alkyl)cycloalkyl, (alkyl)cycloalkenyl, and/or alkylaryl groups. Exemplary substituents include —OH, ═O, —CN, -halo, —NH₂, —NH(C₁-C₄ alkyl), —N(C₁-C₄ alkyl)₂, -phenyl, -phenyl-halo; for example —OH, ═O, —CN, -halo, —NH₂, —NH(C₁-C₄ alkyl), —N(C₁-C₄ alkyl)₂. The backbone of the substituted or unsubstituted lipid may also be interrupted by a disulfide linkage (—S—S—), thioether linkage (—S—), ether linkage —O— or ester (—C(O)O—).

Each of the above terms (e.g., “alkyl,” “cycloalkyl,” “heteroalkyl,” “aryl” and “heteroaryl”), unless otherwise noted, are meant to include both substituted and unsubstituted forms of the indicated radical. Where a substituent is R-substituted (e.g. an RX-substituted alkyl, where “x” is an integer), the substituent may be substituted with one or more R groups as allowed by chemical valency rules where each R group is optionally different (e.g. an RX-substituted alkyl may include multiple R^{x} groups wherein each R^{x} group is optionally different). Certain examples of substituents for each type of radical are provided below.

The term “substituted” as used herein in reference to a moiety means that one or more, especially up to 5, more especially 1, 2 or 3, of the hydrogen atoms in said moiety are replaced independently of each other by the corresponding number of the described substituents. Unless otherwise specified, exemplary substituents include —OH, —CN, —NH₂, —NH(C₁-C₄ alkyl), —N(C₁-C₄ alkyl)₂, ═O, -halo, —C₁-C₆ alkyl, —C₂-C₆ alkenyl, —C₁-C₆ haloalkyl, —C₁-C₆ haloalkoxy and —C₂-C₆ haloalkenyl, —C₁-C₆ alkylcarboxylic acid (e.g. —CH₃COOH or —COOH). Where the substituent is a —C₁-C₆ alkyl or —C₁-C₆ haloalkyl, the C₁-C₆ chain is optionally interrupted by an ether linkage (—O—) or an ester linkage (—C(O)O—). Exemplary substituents for a substituted alkyl may include —OH, —CN, —NH₂, ═O, -halo, —CO₂H, —C₁-C₆ haloalkyl, —C₁-C₆ haloalkoxy and —C₂-C₆haloalkenyl, —C₁-C₆ alkylcarboxylic acid (e.g. —CH₃COOH or —COOH). For example, exemplary substituents for an alkyl may include —OH, —CN, —NH₂, ═O, -halo.

The term “side chain of a natural or non-natural alpha-amino acid” means that any of the groups R^{A} to R^{F} in a natural or non-natural amino acid of formula NH₂—CH(R^{x})—COOH.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, -amino-n-butyric acid, 3,4-dihydroxyphenylalanine, homoserine, α-methylserine, ornithine, pipecolic acid, and thyroxine.

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When any of R^{A} to R^{F} in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified, amino groups may be converted to amides or carbamates, hydroxyl groups may be converted to ethers or esters and thiol groups may be converted to thioethers or thioesters.

Examples of side chains of non-natural alpha amino acids include those referred to below in the discussion of suitable R^{A} to R^{F} groups for use in compounds of the present invention. Salts of the compounds used in the invention include physiologically acceptable acid addition salts for example hydrochlorides, hydrobromides, sulphates, methane sulphonates, p-toluenesulphonates, phosphates, acetates, citrates, succinates, lactates, tartrates, fumarates and maleates. Salts may also be formed with bases, for example sodium, potassium, magnesium, and calcium salts.

There are several chiral centres in the compounds used according to the invention because of the presence of asymmetric carbon atoms. The presence of several asymmetric carbon atoms gives rise to a number of diastereomers with R or S, or D and L stereochemistry at each chiral centre.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Where steric issues determine placement of substituents on a group, the isomer having the lowest conformational energy may be preferred.

Where a compound, moiety, process or product is described as “optionally” having a feature, the disclosure includes such a compound, moiety, process or product having that feature and also such a compound, moiety, process or product not having that feature. Thus, when a moiety is described as “optionally substituted”, the disclosure comprises the unsubstituted moiety and the substituted moiety.

Where two or more moieties are described as being “independently” or “each independently” selected from a list of atoms or groups, this means that the moieties may be the same or different. The identity of each moiety is therefore independent of the identities of the one or more other moieties.

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centres) or double bonds; the racemates, diastereomers, tautomers, geometric isomers and individual isomers are encompassed within the scope of the present invention. The compounds of the present invention do not include those which are known in the art to be too unstable to synthesize and/or isolate.

The symbol [symbol] denotes a point of attachment of a moiety to the remainder of a compound.

The term “prodrug” as used herein represents compounds which are transformed in vivo to the parent compound or other active compound, for example, by hydrolysis in blood. An example of such a prodrug is a pharmaceutically acceptable ester of a carboxylic acid. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996); and The organic chemistry of drug design and drug action by Richard B Silverman in particular pages 497 to 546; each of which is incorporated herein by reference.

The term “pharmaceutical formulation” as used herein includes reference to a formulation comprising at least one active compound and optionally one or more additional pharmaceutically acceptable ingredients, for example a pharmaceutically acceptable carrier. Where a pharmaceutical formulation comprises two or more active compounds, or comprises at least one active compound and one or more additional pharmaceutically acceptable ingredients, the pharmaceutical formulation is also a pharmaceutical composition. Unless the context indicates otherwise, all references to a “formulation” herein are references to a pharmaceutical formulation.

The term “product” or “product of the invention” as used herein includes reference to any product containing a compound of the present invention. In particular, the term product relates to compositions and formulations containing a compound of the present invention, such as a pharmaceutical composition, for example.

The term “therapeutically effective amount” as used herein refers to an amount of a drug, or pharmaceutical agent that, within the scope of sound pharmacological judgment, is calculated to (or will) provide a desired therapeutic response in a mammal (animal or human). The therapeutic response may for example serve to cure, delay the progression of or prevent a disease, disorder or condition.

Preferably, R¹ represents a C₁-C₁₀ alkyl moiety, a C₂-C₁₀, preferably monounsaturated alkenyl moiety or an optionally substituted benzyl moiety. More preferably, R¹ represents one of the following structures:

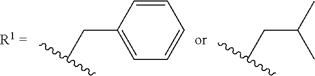

Preferably, R² represents aminoethyl or aminomethyl.

Preferably, R⁴ represents a cycloalkyl moiety having of from 4 to 20 carbon atoms, preferably an alicyclic moiety comprising 5, 6 or 7 carbon atoms, wherein the moiety is a monocyclic, bridged or polycyclic ring preferably selected from the groups consisting of cyclopropyl, cyclobutyl, cyclopentyl, cyclohexyl and cycloheptyl; or a pharmaceutically acceptable salt, solvate, or prodrug thereof. More preferably, R⁴ represents an optionally substituted arylalkyl moiety, an optionally substituted aryl moiety, a biphenyl moiety.

Preferably, (*) denominates the stereochemistry at the indicated carbon atom, wherein each may independently be L or D.

The present invention also relates to a process for the preparation of a compound according to the invention, comprising the steps of a) removing the side chain from polymyxin B by enzymatic digestion with ficin to obtain a polymyxin nonapeptide macrocyle comprising a side chain having a free N-terminal amine group; b) protecting the amino functions of the ring, leaving the N-terminal amine unprotected to obtain a polymyxin nonapeptide macrocyle comprising 4 protected amine groups and a free N-terminal amine group, and c) coupling the free N-terminal amine group with a disulfide containing compound, to obtain a disulfide-coupled polymyxin nonapeptide macrocyle comprising 4 protected amine groups, and d) removing the protection groups and isolating the compound of formula (1).

The present invention also relates to a process for the preparation of a compound according to the invention, comprising the steps of protecting the amino function of polymyxin B to obtain an N-protected polymyxin B; removing the complete side chain from by enzymatic digestion from the N-protected polymyxin B with savine to obtain a tri-N-protected polymyxin B heptameric macrocycle with a single free amino group; coupling the free amino group with a disulfide lipidated tripeptide or N-terminal amide or carbamate linkage building block, to obtain a disulfide-coupled polymyxin nonapeptide macrocyle comprising 3 protected amine groups, and removing the protection groups and isolating the compound of formula (1).

The present invention also relates to a process for the preparation of a compound according to the invention, wherein the protective group is a tert-Butyloxycarbonyl (BOC) protecting group.

The present invention also relates to a process for the preparation of a compound according to the invention, wherein the lipidated tripeptide building block, preferably the disulfide lipidated tripeptide building block, is prepared using a solid phase peptide synthesis.

The present invention also relates to a process for the preparation of a compound according to the invention, wherein the disulfide lipidated N-terminal amide or carbamate linkage building block has a structure according to formula (VIIa) or (VIIb):

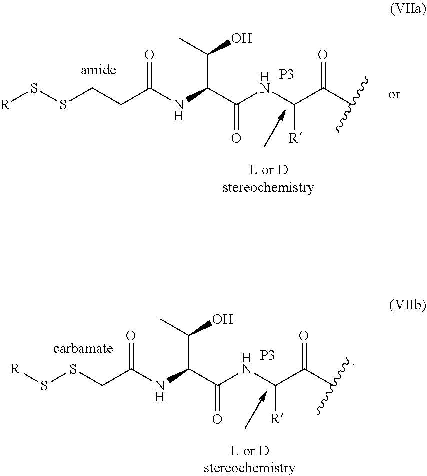

The present invention also relates to a process for the preparation of a compound according to the invention, comprising the steps of:
a) removing the side chain from a polymyxin by enzymatic digestion with an enzyme capable of disrupting the bond between the exocyclic amino acid positioned nearest to the fatty acid tail and the middle exocyclic amino acid to obtain a polymyxin nonapeptide macrocycle comprising a side chain having a free N-terminal amine group;
b) protecting the amino functions of the ring, leaving the N-terminal amine unprotected to obtain a polymyxin nonapeptide macrocycle comprising protected amine groups and a free N-terminal amine group;
c) coupling the free N-terminal amine group with a compound, to obtain a coupled polymyxin nonapeptide macrocycle comprising protected amine groups; and
d) removing the protection groups and isolating the respective obtained compound of formula (I), (Ia), (II), (IIa), (IIb), (III), (IIIa), (IV), (V), or (VI),
preferably, wherein the polymyxin nonapeptide macrocycle comprises 4 protected amine groups and a free N-terminal amine group, and the coupled polymyxin nonapeptide macrocycle comprises 4 or 5 protected amine groups; and/or
preferably, wherein step c comprises coupling the free N-terminal amine group with a disulfide containing compound, to obtain a disulfide coupled polymyxin nonapeptide macrocycle comprising 4 or 5 protected amine groups.

The present invention also relates to a process for the preparation of a compound according to the invention, comprising the steps of:
a) protecting the amino functions of a polymyxin to obtain an N-protected polymyxin;
b) removing the complete side chain by enzymatic digestion from the N-protected polymyxin with an enzyme capable of disrupting the bond between the exocyclic amino acid positioned nearest to the cyclic heptapeptide and the cyclic heptapeptide to obtain a N-protected polymyxin heptameric macrocycle with a single free amino group;
c) coupling the free amino group with a lipidated tripeptide or N-terminal amide or carbamate linkage building block, to obtain a coupled polymyxin peptide macrocycle comprising protected amine groups, and
d) removing the protection groups and isolating the respective obtained compound of formula (I), (Ia), (II), (IIa), (IIb), (III), (IIIa), (IV), (V), or (VI),
preferably, wherein the N-protected polymyxin heptameric macrocycle is tri-N-protected, and the coupled polymyxin peptide macrocycle comprises 3, 4, or 5, preferably 4 or 5 or preferably 3, protected amine groups; and/or
preferably, wherein step c comprises coupling the free amino group with a disulfide lipidated tripeptide or N-terminal amide or carbamate linkage building block, to obtain a disulfide-coupled polymyxin nonapeptide macrocycle comprising 3 protected amine groups.

Preferably, the coupled polymyxin peptide macrocycle is a coupled polymyxin nonapeptide or a coupled polymyxin decapeptide.

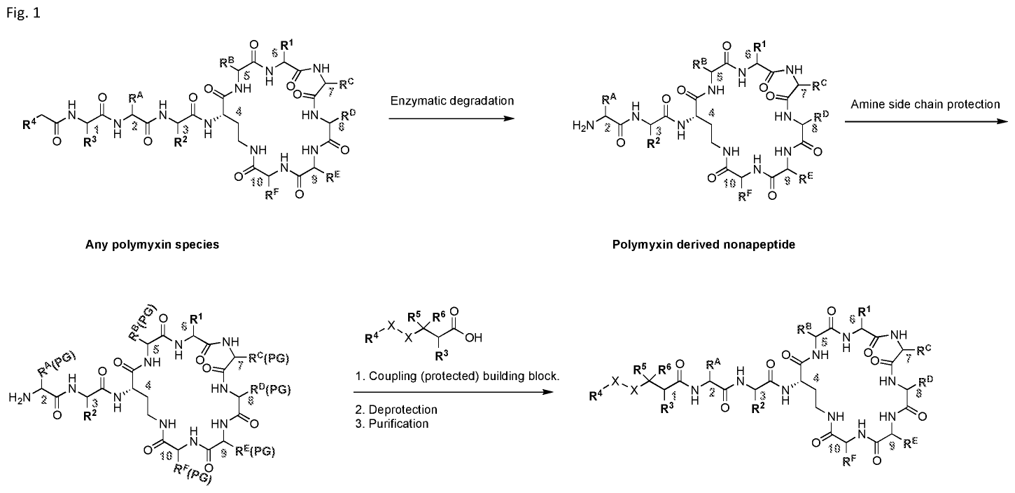

With reference to FIGS. 1 to 8, the term ‘polymyxin nonapeptide macrocycle’ herein is understood to mean that the polymyxin compound comprises nine amino acids and a macrocycle. For the avoidance of doubt, this does not imply that the macrocycle comprises nine amino acids incorporated into the macrocycle, but refers to the total number of amino acids.

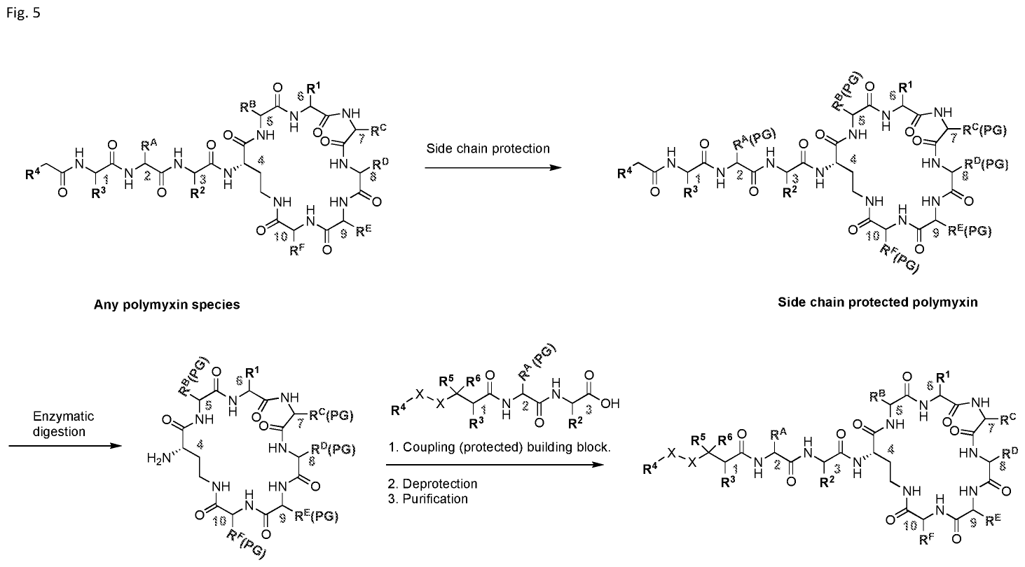

Accordingly, with reference to FIGS. 5 to 8, the term ‘polymyxin decapeptide macrocycle’ herein is understood to mean that the polymyxin compound comprises ten amino acids and a macrocycle.

The present invention also relates to a process for the preparation of a compound according to the invention, wherein the polymyxin is polymyxin B or polymyxin E.

The present invention also relates to a process for the preparation of a compound according to the invention, wherein the enzyme is a hydrolytic enzyme, preferably a proteolytic enzyme, more preferably ficin, savinase, or subtilisin.

Preferably, in the process according to the invention, the bond is a peptide bond.

The present invention also relates to a process for the preparation of a compound according to the invention, wherein the disulfide containing compound has a structure according to structure BXXIII or BXXIV:

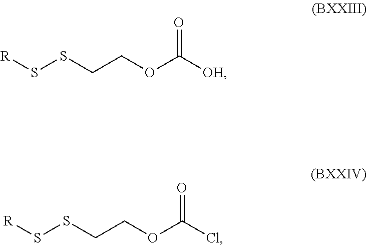

wherein R represents an optionally substituted alkyl moiety, an optionally substituted cycloalkyl moiety; or an optionally substituted aryl moiety, preferably wherein R is an optionally substituted alkyl moiety or an optionally substituted aryl moiety.

The present invention also relates to a process for the preparation of a compound according to the invention, wherein the disulfide lipidated N-terminal amide or carbamate linkage building block has a structure according to structure BXXV or BXXVI:

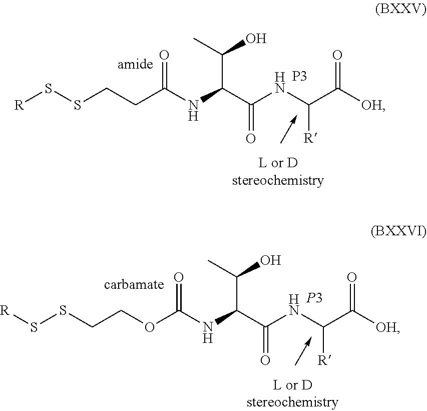

wherein R represents an optionally substituted alkyl moiety, an optionally substituted cycloalkyl moiety; or an optionally substituted aryl moiety, preferably wherein R is an optionally substituted alkyl moiety or an optionally substituted aryl moiety; and
wherein R′ represents hydrogen, hydroxymethyl, aminoethyl, aminomethyl, or 5-(2-amino)pentanoic acid.

Preferably, R¹ together with the carbonyl group and nitrogen in α-position to the carbon to which it is attached, represents D-phenylalanine or D-leucine. More preferably, R¹ together with the carbonyl group and nitrogen in α-position to the carbon to which it is attached, preferably represents D-phenylalanine. Where in the following or above reference is made to amino acids, these are represented by the substituent R¹ to R⁴, and the adjacent atoms forming an amino acid moiety. E.g. where R¹ together with the carbonyl group and nitrogen alpha to the carbon to which it is attached, represents D-phenylalanine, R¹ represents a benzyl group. In this case the amino acid residue found at position 6 in formula I, Ia, II, IIa, Ill, IV, V, or VI represents D-Phe:

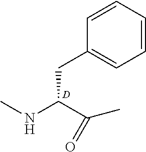

Where R¹ together with the carbonyl group and nitrogen alpha to the carbon to which it is attached, represents, R¹ is an isopropyl group. In this case the amino acid residue found at position 6 is D-Leu in formula I, Ia, II, IIa, III, IV, V, or VI has the following structure:

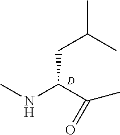

### Compounds

In one aspect, the invention provides compounds of formula (1) as previously described or a pharmaceutically acceptable salt, stereoisomer, solvate or prodrug thereof.

In another aspect, the invention provides compounds of formula (II) as previously described or a pharmaceutically acceptable salt, stereoisomer, solvate or prodrug thereof.

In another aspect, the invention provides compounds of formula (III) as previously described or a pharmaceutically acceptable salt, stereoisomer, solvate or prodrug thereof.

In yet another aspect, the invention provides compounds of formula (IV) as previously described or a pharmaceutically acceptable salt, stereoisomer, solvate or prodrug thereof.

In yet another aspect, the invention provides compounds of formula (V) as previously described or a pharmaceutically acceptable salt, stereoisomer, solvate or prodrug thereof.

In yet another aspect, the invention provides compounds of formula (VI) as previously described or a pharmaceutically acceptable salt, stereoisomer, solvate or prodrug thereof.

### Formulations and Administration

According to a further aspect of the invention there is provided a pharmaceutical formulation or composition including a compound of the invention, optionally in admixture with at least one pharmaceutically acceptable adjuvant, diluent or carrier.

The formulation or composition may be a parenteral formulation or an oral formulation. The formulation may be a parenteral formulation, for example a formulation for intravenous injection. The formulation may be an oral formulation.

Compounds, formulations or compositions of the invention may be administered orally, topically, intravenously, subcutaneously, buccally, rectally, dermally, nasally, tracheally, bronchially, by any other parenteral route, as an oral or nasal spray or via inhalation. The compounds may be administered in the form of pharmaceutical preparations comprising the compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Typically, therefore, the pharmaceutical compounds of the invention may be administered parenterally (“parenterally” as used herein, refers to modes of administration which include intravenous, intramuscular, intraperitoneal, intrasternal, subcutaneous and intraarticular injection and infusion) or orally to a host to obtain an antibacterial effect. For example, the pharmaceutical compounds of the invention may be administered by intravenous injection or infusion. In the case of larger animals, such as humans, the compounds may be administered alone or as compositions in combination with pharmaceutically acceptable diluents, excipients or carriers.

Actual dosage levels of active ingredients in the pharmaceutical formulations and pharmaceutical compositions of this invention may be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved. Suitable doses are generally in the range of from 0.01-100 mg/kg/day, for example in the range of 0.1 to 50 mg/kg/day.

Pharmaceutical formulations or compositions of this invention for parenteral (e.g. intravenous) injection may comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters, such as ethyl oleate. Proper fluidity can be maintained, for example, by the use of coating materials, such as lecithin, by the maintenance of the required particle size in the case of dispersions and by the use of surfactants. Formulations or compositions for parenteral injection may represent preferred formulations or compositions of the invention.

These compositions may also contain adjuvants such as preservative, wetting agents, emulsifying agents and dispersing agents. Inhibition of the action of microorganisms may be ensured by the inclusion of various antibacterial and antifungal agents, for example, paraben, chlorobutanol or phenol sorbic acid. It may also be desirable to include isotonic agents, such as sugars or sodium chloride, for example. Prolonged absorption of the injectable pharmaceutical form may be brought about by the inclusion of agents (for example, aluminium monostearate and gelatine) which delay absorption.

Solid dosage forms for oral administration include capsules, tablets, pills, powders and granules. In such solid dosage forms, the active compound is typically mixed with at least one inert, pharmaceutically acceptable excipient or carrier, such as sodium citrate or dicalcium phosphate and/or one or more: a) fillers or extenders, such as starches, lactose, sucrose, glucose, mannitol and silicic acid; b) binders, such as carboxymethylcellulose, alginates, gelatin, polyvinylpyrrolidone, sucrose and acacia; c) humectants, such as glycerol; d) disintegrating agents, such as agar-agar, calcium carbonate, potato or tapioca starch, alginic acid, certain silicates and sodium carbonate; e) solution retarding agents, such as paraffin; f) absorption accelerators, such as quaternary ammonium compounds; g) wetting agents, such as cetyl alcohol and glycerol monostearate; h) absorbents, such as kaolin and bentonite clay and i) lubricants, such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Oral formulations may contain a dissolution aid. Examples of dissolution aids include nonionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkyamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsufonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

The active compounds may also be in microencapsulated form, if appropriate, with one or more of the above-mentioned excipients.

The active compounds may be in finely divided form, for example it may be micronized.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavouring and perfuming agents. Suspensions, in addition to the active compounds, may contain suspending agents such as ethoxylated isostearyl alcohols, polyoxyethylene sorbitol and sorbitan esters, microcrystalline cellulose, aluminium metahydroxide, bentonite, agar-agar, and tragacanth and mixtures thereof.

Compositions for rectal or vaginal administration may be in the form of suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, creams, foams, gels, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Liquid (e.g. aqueous) formulations and compositions, whether intended for parenteral or oral use, may comprise additional compound(s) to help prevent precipitation of the active compound. Compounds of the invention are glycopeptide derivatives. Precipitation of such compounds in aqueous solution may be avoided or minimised by including a monosaccharide in the solution. For example, aqueous formulations or compositions may comprise glucose. In particular, a parenteral (e.g. intravenous injection) formulation or composition may comprise a compound of the invention, water for injection and glucose.

Insofar as they do not interfere with the activity of the compounds, the formulations or compositions according to the present subject matter may contain other active agents intended, in particular, for use in treating a bacterial infection.

The formulations according to the present subject matter may also contain inactive components. Suitable inactive components are well known in the art and are described in standard textbooks, such as Goodman and Gillman's: The Pharmacological Bases of Therapeutics, 13^{th} Ed., Brunton et al., Eds. McGraw-Hill Education (2017), and Remington's Pharmaceutical Sciences, 17^{th} Ed., Mack Publishing Co., Easton, Pa. (1990), both of which are incorporated by reference herein in their entirety.

The formulations may be used in combination with an additional pharmaceutical dosage form to enhance their effectiveness in treating any of the disorders described herein. In this regard, the present formulations may be administered as part of a regimen additionally including any other pharmaceutical and/or pharmaceutical dosage form known in the art as effective for the treatment of any of these disorders.

### Uses

The compounds of the invention represent a novel class of polymyxin or polymyxin derivatives. Polymyxins, especially polymyxin B and Colistin are antibiotics that are active against Gram-negative bacteria.

Compounds provided herein represent antibiotics, in particular antibiotics useful for the treatment of conditions related to infection by Gram-negative bacteria. The compounds of the invention may provide similar or better activity, while showing a lower nephrotoxicity.

The compounds are preferably used for the treatment of a bacterial infection, the bacterial infection may be caused by Gram-negative or Gram-positive bacteria. For example, the bacterial infection may be caused by bacteria from one or more (e.g. at least one) of the following families: Clostridium, Pseudomonas, Escherichia, Klebsiella, Enterococcus, Enterobacter Serratia, Stenotrophomonas, Aeromonas, Morganella, Yersinia, Salmonella. Proteus, Pasteurella, Haemophilus, Citrobacter, Burkholderia, Brucella, Moraxella, Mycobacterium, Streptococcus or Staphylococcus. Particular examples include Clostridium, Pseudormonas, Escherichia, Klebsiella, Enterococcus, Enterobacter, Streptococcus and Staphylococcus. The bacterial infection may, for example, be caused by one or more bacteria selected from Moraxella catarrhalis, Brucella abortus, Burkholderia cepacia, Citrobacter species, Escherichia coli, Haemophilus pneumonia, Klebsiella Pneumonia, Pasteureila multocida, Proteus mirabilis, Salmonella typhimurium, Clostridium difficile. Yersinia enterocolitica Mycobacterium tuberculosis. Staphylococcus aureus, group B streptococci, Streptococcus Pneumonia, and Streptococcus pyogenes.

The compounds of the invention are particularly useful for the treatment of bacterial infection caused by Gram-negative bacteria.

### Assays

Compounds of the invention can be assessed for biological activity using any suitable assay that would be known to the person skilled in the art. Exemplary assays that are useful for the assessment of compounds of the invention are provided in the following paragraphs.

The antibacterial activity of the compounds was tested against a panel of bacteria, including Gram-negative bacteria. A panel of obtained compounds is summarized in Tables A to C below, showing the structure and efficacies. Particularly preferred compounds include those listed in the following Tables 1 to 6:

TABLE 1

| Substituent | Amino Acid Formed | Amino Acid Formed |
|---|---|---|
| R¹ | D-Phe or D-Leu | D-Phe |
| R² | Dab (L/D) or Dap (L/D) | Dab (L/D) or Dap (L/D) |
| | or Ser (D) or Gly | |
| N-terminal Cysteine | D | D |
| stereochemistry | | |
| R³ | 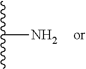 [SMILES: *N] | 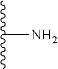 [SMILES: *N] |
| | 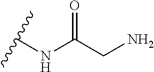 [SMILES: *NC(=O)CN] | |
| R⁴ | 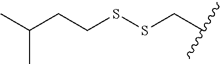 [SMILES: *CSSCCC(C)C] | 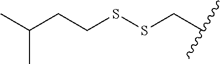 [SMILES: *CSSCCC(C)C] |

TABLE 2

| Rest | Amino Acid Formed | Amino Acid Formed |
|---|---|---|
| R¹ | D-Phe or D-Leu | D-Phe |
| R² | Dab (L) | Dab (L) |
| N-terminal Cysteine | L | L |
| stereochemistry | | |
| R³ | 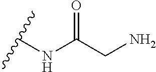 [SMILES: *NC(=O)CN] | 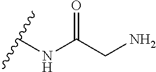 [SMILES: *NC(=O)CN] |
| R⁴ | 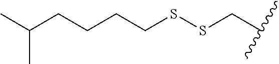 [SMILES: *CSSCCCCC(C)C] | 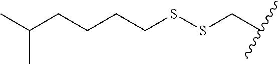 [SMILES: *CSSCCCCC(C)C] |

TABLE 3

| Rest | Amino Acid Formed | Amino Acid Formed |
|---|---|---|
| R¹ | D-Phe or D-Leu | D-Phe |
| R² | Dab (L) | Dab (L) |
| N-terminal Cysteine | L | L |
| stereochemistry | | |
| R³ | 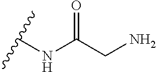 [SMILES: *NC(=O)CN] | 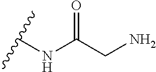 [SMILES: *NC(=O)CN] |
| R⁴ | 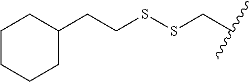 [SMILES: *CSSCCC1CCCCC1] | 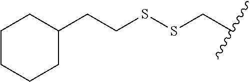 [SMILES: *CSSCCC1CCCCC1] |

TABLE 4

| Rest | Amino Acid Formed | Amino Acid Formed |
|---|---|---|
| R¹ | D-Phe or D-Leu | D-Phe |
| R² | Dab (D) or Dap (L/D) | Dab (D) or Dap (D) |
| | or Gly | or Gly |
| N-terminal Cysteine | D | D |
| stereochemistry | | |
| R³ | 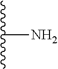 [SMILES: *N] | 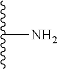 [SMILES: *N] |
| R⁴ | 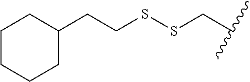 [SMILES: *CSSCCC1CCCCC1] | 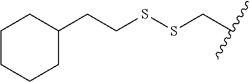 [SMILES: *CSSCCC1CCCCC1] |

TABLE 5

| Rest | Amino Acid Formed | Amino Acid Formed |
|---|---|---|
| R¹ | D-Phe or D-Leu | D-Phe |
| R² | Dab (D) or Dap (D) | Dab (D) or Dap (D) |
| N-terminal Cysteine | D | D |
| stereochemistry | | |
| R³ | 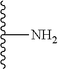 [SMILES: *N] | 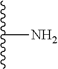 [SMILES: *N] |
| R⁴ | 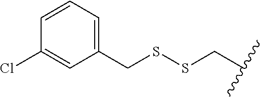 [SMILES: *CSSCc1cccc(Cl)c1] | 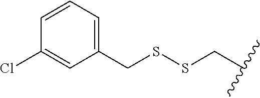 [SMILES: *CSSCc1cccc(Cl)c1] |

TABLE 6

| | |
|---|---|
| R¹ | D-Phe or D-Leu |
| R² | Dab (L) |
| N-terminal Cysteine | D |
| stereochemistry | |
| R³ | 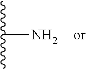 [SMILES: *N] |
| | 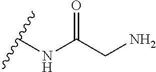 [SMILES: *NC(=O)CN] |
| R⁴ | 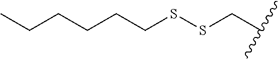 [SMILES: *CSSCCCCCC] |

Especially preferred compounds include those listed in the following Table 7.

TABLE 7

| Example | |
|---|---|
| compound | Structure |
| 1 | 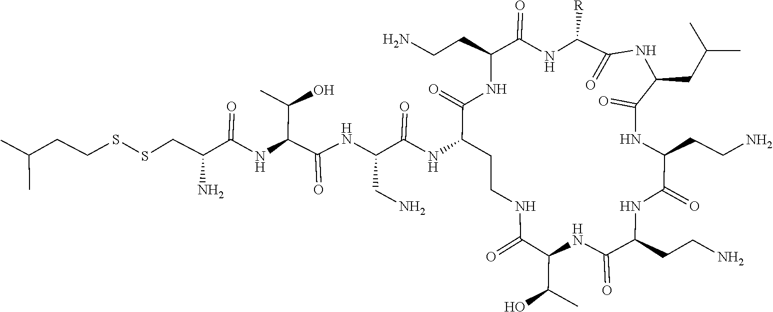 |
| 2 | 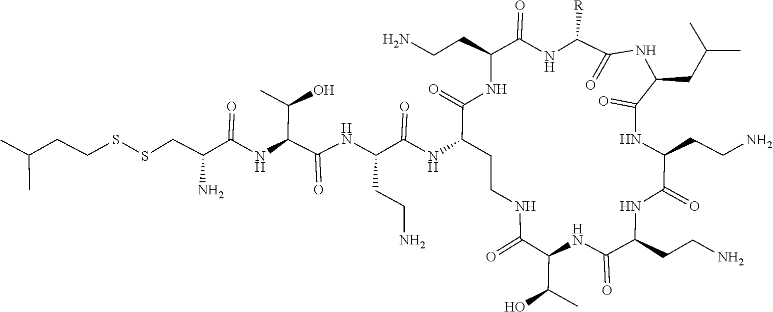 |
| 3 | 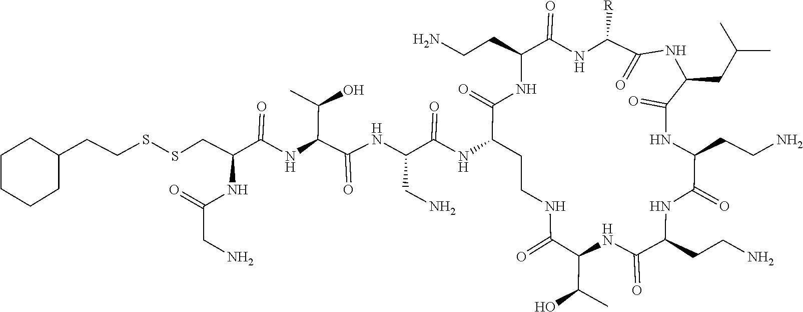 |
| 4 | 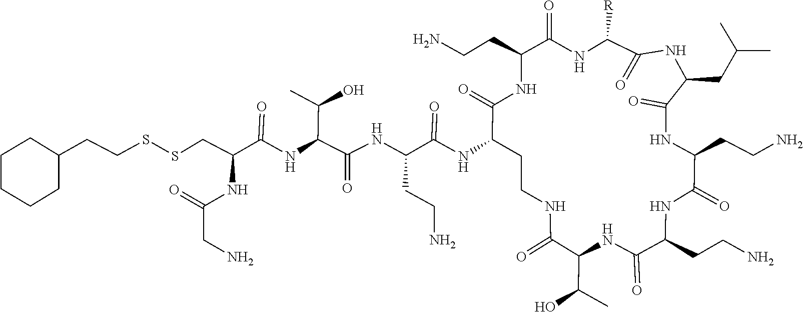 |
| 5 | 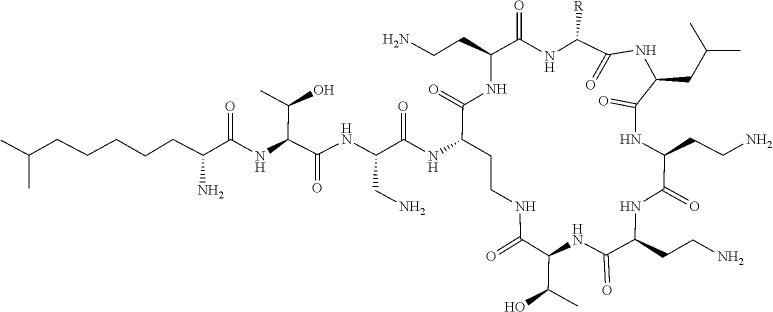 |
| 6 | 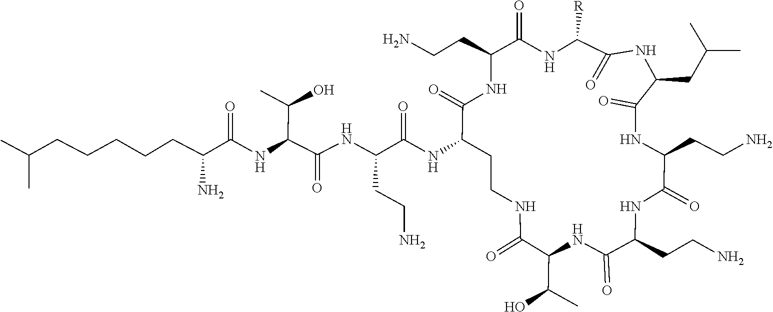 |
| 7 | 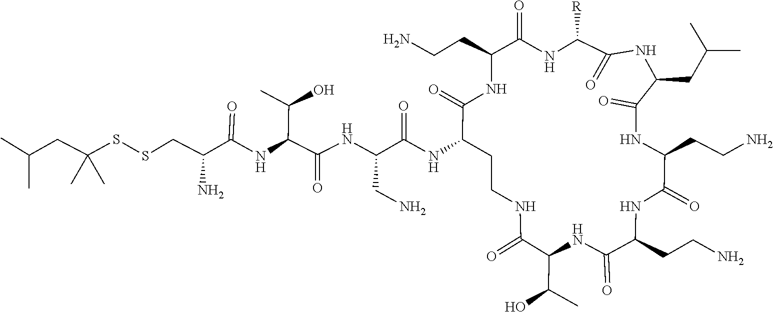 |

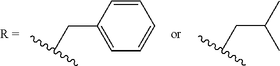

The example compounds of Table 7 can be described as having the following features: 1. Diaminopropionic acid (Dap) at P3, 2. Diaminobutyric acid (Dab) at P3, 3. Diaminopropionic acid (Dap) at P3, 4. Diaminobutyric acid (Dab) at P3, 5. All carbon lipid variant with Dap at P3, 6. All carbon lipid variant with Dab at P3, 7. Lipid with substitution adjacent to disulfide motif and Dap at P3.

### Synthesis of Compounds

Compounds of the invention can be made according to reaction schemes set out herein below:

Scheme 1 (FIG. 1) shows the general preparation procedure via polymyxin nonapeptide, namely the preparation by a synthesis of polymyxin analogues starting from any polymyxin species via polymyxin nonapeptide.

In FIG. 1, R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with hydroxyl, suflhydryl, alkyl thiol ethers, carboxyl, preferably β-carboxyl or γ-carboxyl, aromatic or heteroaromatic substituents, preferably benzyl, guanidinium or imidazolium, and/or amino groups, preferably ε-NH₃⁺;

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety; each X independently represents C, S, O, or N.

Further, “PG” indicates protected group, i.e. protected amine side chains of R^{A}; R^{B}; R^{C}; R^{D}; R^{E}; or R^{F}. Exemplary building blocks are outlined below.

Enzymatic degradation in Scheme 1 may be effected by an enzyme capable of cleaving the polymyxin between positions 1 and 2, preferably wherein the enzyme is a hydrolytic enzyme, preferably wherein the enzyme is ficin.

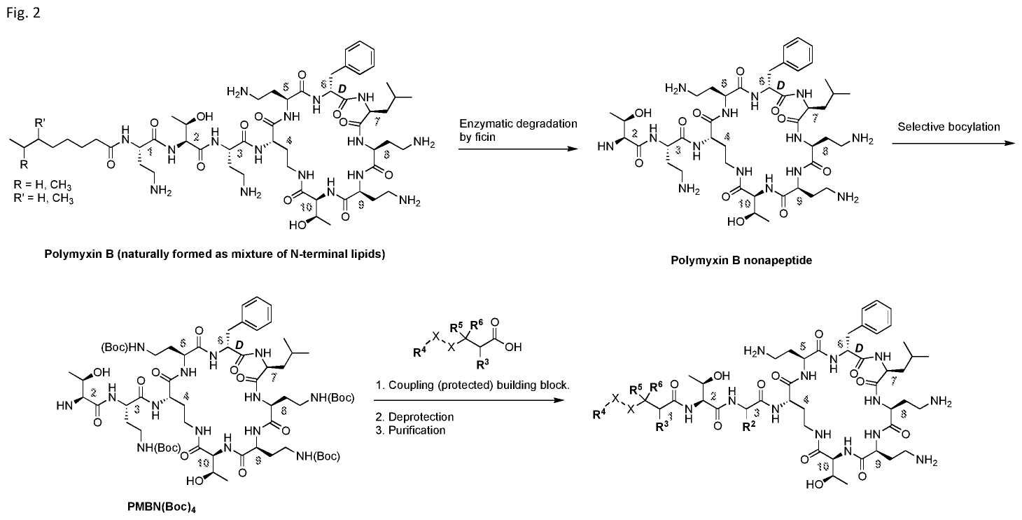

Scheme 2 (FIG. 2) shows the specific preparation procedure via polymyxin B nonapeptide (PMBN), namely the preparation by a synthesis of polymyxin B analogues starting from polymyxin B via polymyxin B nonapeptide.

In FIG. 2, R² represents aminoethyl; R³ represents —NH₂ or —N(H)—COCH₂NH₂;

R⁴ represents an optionally substituted alkyl moiety, an optionally substituted cycloalkyl moiety; or an optionally substituted aryl moiety; R⁵ and R⁶ each independently represent hydrogen or an optionally substituted alkyl moiety; each X independently represents C, S, O, or N.

Exemplary building blocks are outlined below.

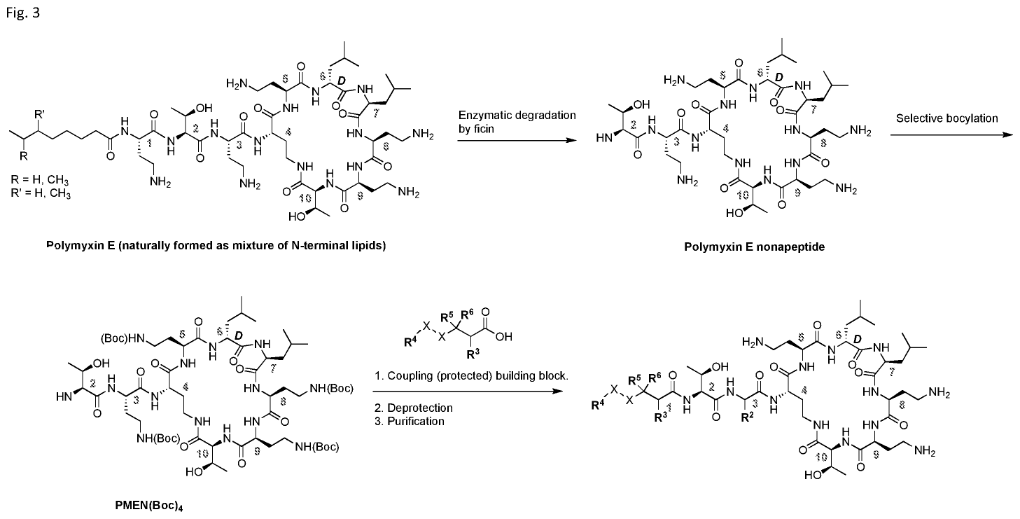

Scheme 3 (FIG. 3) shows the specific preparation procedure via polymyxin E nonapeptide (PMEN), namely the preparation by a synthesis of polymyxin E analogues starting from polymyxin E via polymyxin E nonapeptide.

In FIG. 3, R² represents aminoethyl; R³ represents —NH₂ or —N(H)—COCH₂NH₂;

R⁴ represents an optionally substituted alkyl moiety, an optionally substituted cycloalkyl moiety; or an optionally substituted aryl moiety; R⁵ and R⁶ each independently represent hydrogen or an optionally substituted alkyl moiety; each X independently represents C, S, O, or N.

Exemplary building blocks are outlined below.

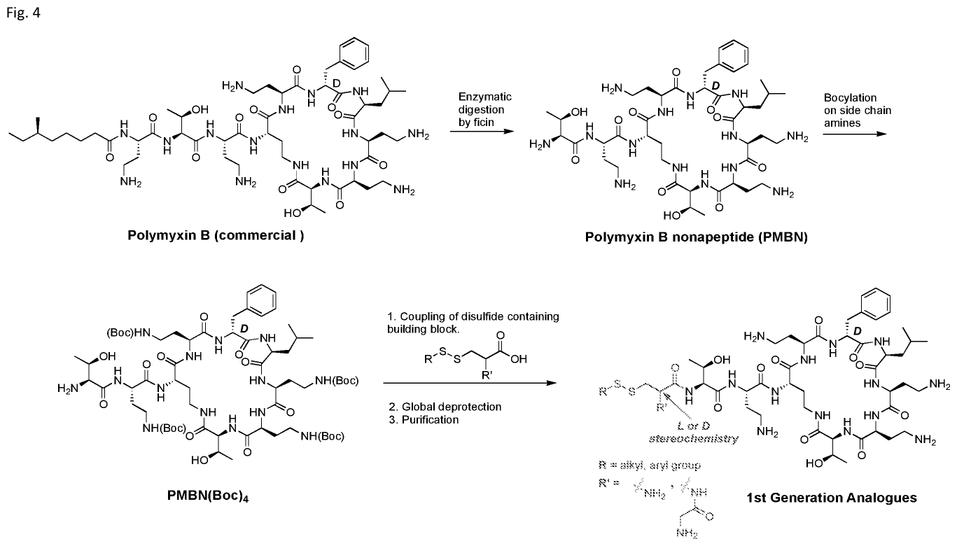

Scheme 4 (FIG. 4) shows the preparation by a synthesis of polymyxin analogues containing a disulfide tail, starting from commercially available polymyxin B. The disulfide lipid building blocks are outlined below.

A number of building blocks were required for the synthesis of these polymyxin analogues according to any one of Scheme 1 to 4. As illustrated herein below, a first series of disulfide containing building blocks prepared consist of simple aliphatic groups and are derivatives of either D-cysteine (D-Cys) or L-cysteine (L-Cys). Variation is found in the lipophilic alkyl tail and in the level of substitution on the amine. Both Boc-protected compounds (BI to BIV) and compounds bearing an additional Gly motif (BV to BVIII) were prepared.

The following compounds are cysteine-based disulfide building blocks with aliphatic tails, useful disulfide lipidated tripeptide building blocks having the structures BI to BIX, to couple to any polymyxin species enzymatically degraded to a nonapeptide followed by amine side chain protection according to any one of Scheme 1 to 4, for example PMEN(Boc)₄ or PMBN(Boc)₄:

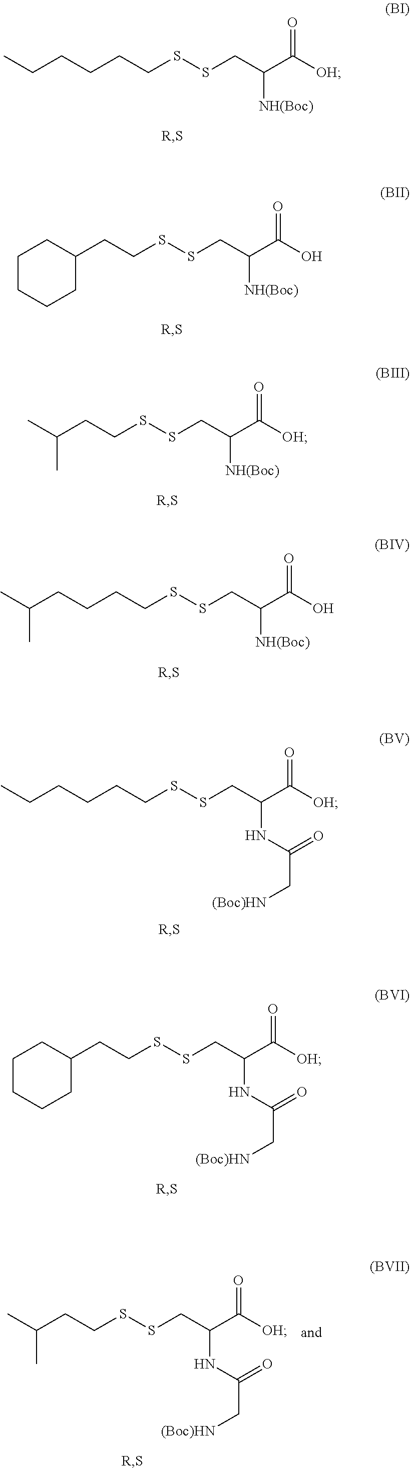

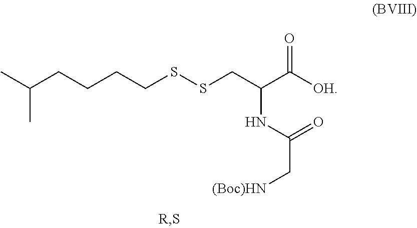

Furthermore, compounds with an aromatic substituted tail on the Cys scaffold were synthesized, according to structures BIX to BXV, from top left to right to bottom row):

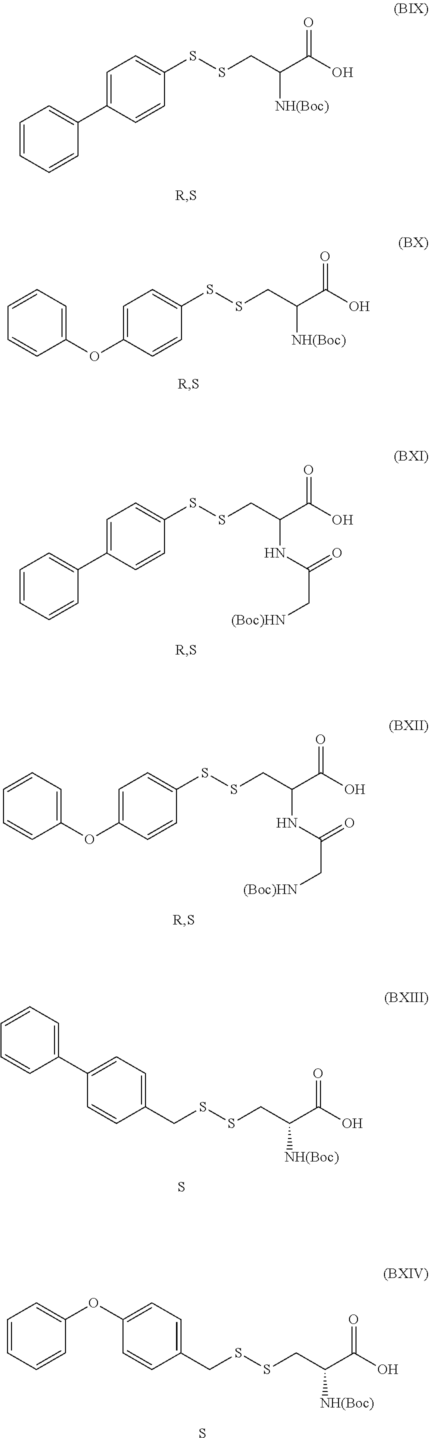

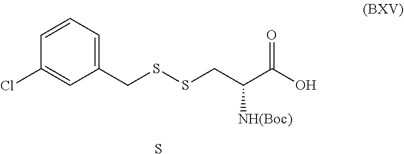

Herein, the aromatic moiety and the linker between the aromatic moiety and the thiol were varied, either directly connected as in 4-phenoxybenzenethiol, or connected via an extra methylene linker as in 4-phenoxyphenyl)methanethiol.

A non-disulfide containing analogue was prepared as well, and in addition, an analogue with D-penicillamine (D-Pen) instead of D-Cys was prepared (BXVI and BXVII):

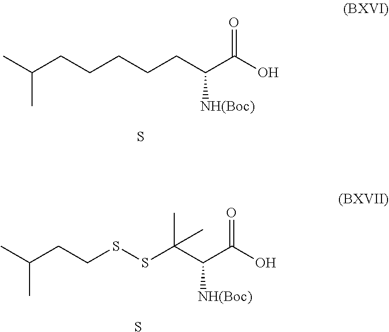

Schemes 1-4 are used to generate so-called 1^{st} generation compounds.

Scheme 5 (FIG. 5) shows a separate general synthesis route of compounds according to the invention via polymyxin heptapeptide including P3 variations, namely the preparation by a synthesis of polymyxin analogues starting from any polymyxin species via polymyxin heptapeptide.

In FIG. 5, R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with hydroxyl, suflhydryl, alkyl thiol ethers, carboxyl, preferably β-carboxyl or γ-carboxyl, aromatic or heteroaromatic substituents, preferably benzyl, guanidinium or imidazolium, and/or amino groups, preferably ε-NH₃⁺;

R¹ represents an optionally substituted alkyl moiety, an optionally substituted benzyl moiety;

R² represents hydrogen, hydroxymethyl, aminoethyl, aminomethyl, or 5-(2-amino)pentanoic acid;

R³ represents —NH₂ or —N(H)—COCH₂NH₂;

R⁴ represents an optionally substituted alkyl moiety, an optionally substituted cycloalkyl moiety; or an optionally substituted aryl moiety;

R⁵ and R⁶ each independently represent hydrogen or an optionally substituted alkyl moiety; each X independently represents C, S, O, or N.

Further, “PG” indicates protected group, i.e. protected amine side chains of R^{A}; R^{B}; R^{C}; R^{D}; R^{E}; or R^{F}.

Enzymatic degradation in Scheme 5 may be effected by an enzyme capable of cleaving the polymyxin between positions 3 and 4, preferably wherein the enzyme is a hydrolytic enzyme, preferably wherein the enzyme is savinase. The term ‘savinase’ is a trademark. Savinase is known under several names, for example subtilisin, and is indexed by the International Union of Biochemistry and Molecular Biology (IUBMB) as EC 3.4.21.62.

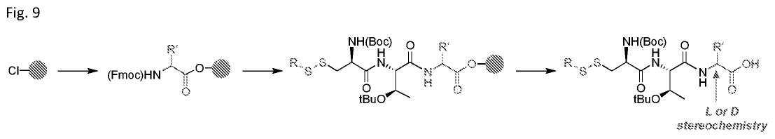

Compounds are prepared starting from commercially available polymyxin. After protection of amine side chains and enzymatic degradation, a protected polymyxin heptapeptide is obtained, which is further conjugated to separately synthesized building blocks in a convergent synthesis, see FIG. 9 showing Scheme 9 for an example of the preparation thereof.

The building blocks can be synthesized using solid phase peptide synthesis, as set out in FIG. 9/Scheme 9, showing a representative synthesis of trimeric peptide building blocks used in the preparation of exemplary second generation polymyxin analogues. CTC resin is substituted with the desired amino acid, conjugated via its carboxylic acid. Standard solid phase peptide synthesis (SPPS) procedures yield the desired peptide on resin. This allows for convenient variation of the P3 amino acid as well as optional introduction of a disulfide linked lipid tail. In this set of lipidated tripeptides the N-terminal cysteine is D-Cys. The lipids or disulfide lipids used in the synthesis of the 2^{nd} generation analogues can be selected from the compounds shown above for the 1^{st} generation compounds.

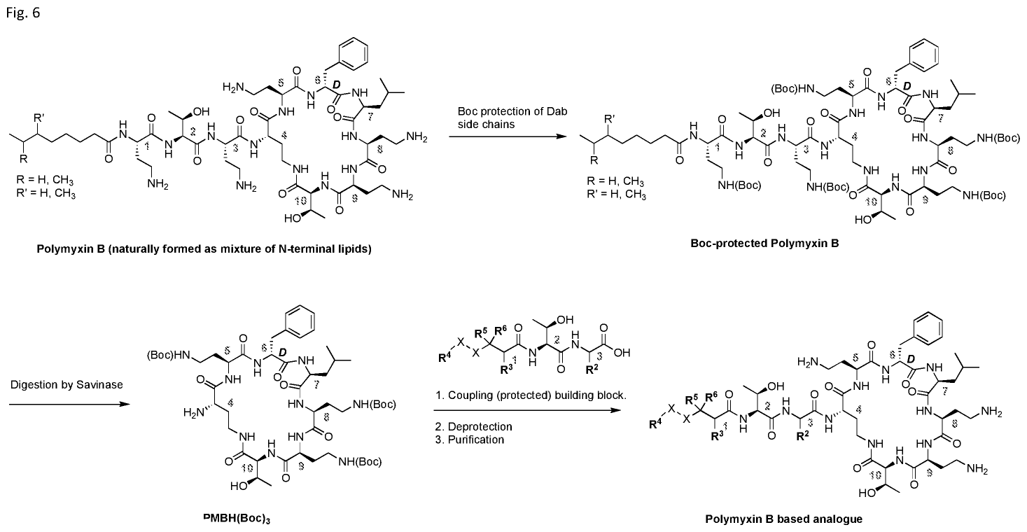

Scheme 6 (FIG. 6) shows the specific preparation procedure via polymyxin B heptapeptide (PMBH) including P3 variations, namely the preparation by a synthesis of polymyxin B analogues starting from polymyxin B via polymyxin B heptapeptide.

In FIG. 6, R² represents hydrogen, hydroxymethyl, aminoethyl, aminomethyl, or 5-(2-amino)pentanoic acid; R³ represents —NH₂ or —N(H)—COCH₂NH₂;

R⁴ represents an optionally substituted alkyl moiety, an optionally substituted cycloalkyl moiety; or an optionally substituted aryl moiety; R⁵ and R⁶ each independently represent hydrogen or an optionally substituted alkyl moiety; each X independently represents C, S, O, or N.

Compounds are prepared starting from commercially available polymyxin B. After Bocylation and enzymatic digestion by savinase, tri-Boc-protected polymyxin B heptapeptide (PMBH(Boc)3) is obtained, which is further conjugated to the separately synthesized building blocks in a convergent synthesis, see FIG. 9 showing scheme 9 for an example of the preparation thereof.

These so-called 2^{nd} generation analogues are prepared as indicating above in Scheme 6. Commercially available polymyxin B, is first treated with Boc-anhydride, yielding the polymyxin species with all free amines Boc-protected. This protected species is subjected to enzymatic digestion by the industrial enzyme savinase, yielding the heptameric macrocycle PMBH(Boc)₃. The preparation of PMBH(Boc)₃ is well-known from literature. Subsequent coupling to a building block, preferably a lipidated tripeptide building block or a building block comprising two amino acids that are connected to a lipid tail with an alpha-amine, followed by global deprotection and purification provides the 2^{nd} generation analogues.

The lipidated tripeptide building blocks were synthesized using solid phase peptide synthesis, as set out in FIG. 9/Scheme 9, showing a representative synthesis of trimeric peptide building blocks used in the preparation of exemplary second generation polymyxin analogues. CTC resin is substituted with the desired amino acid, conjugated via its carboxylic acid. Standard solid phase peptide synthesis (SPPS) procedures yield the desired peptide on resin. This allows for convenient variation of the P3 amino acid as well as optional introduction of a disulfide linked lipid tail. In this set of lipidated tripeptides the N-terminal cysteine is D-Cys. The lipids or disulfide lipids used in the synthesis of the 2^{nd} generation analogues can be selected from the compounds shown above for the 1^{st} generation compounds.

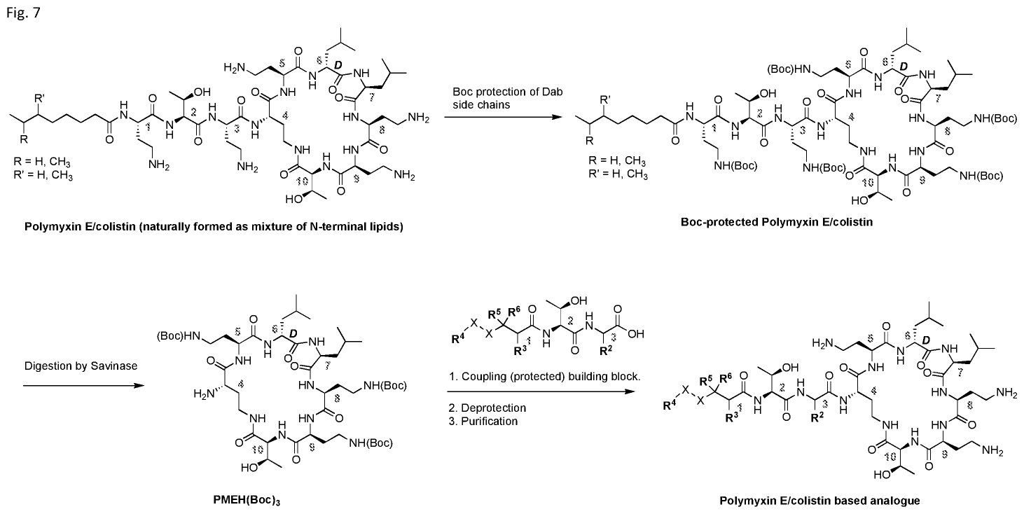

Scheme 7 (FIG. 7) shows the specific preparation procedure via polymyxin E heptapeptide (PMEH) including P3 variations, namely the preparation by a synthesis of polymyxin E analogues starting from polymyxin E via polymyxin E heptapeptide.

In FIG. 7, R² represents hydrogen, hydroxymethyl, aminoethyl, aminomethyl, or 5-(2-amino)pentanoic acid; R³ represents —NH₂ or —N(H)—COCH₂NH₂;

R⁴ represents an optionally substituted alkyl moiety, an optionally substituted cycloalkyl moiety; or an optionally substituted aryl moiety; R⁵ and R⁶ each independently represent hydrogen or an optionally substituted alkyl moiety; each X independently represents C, S, O, or N.

Compounds are prepared starting from commercially available polymyxin E. After Bocylation and enzymatic digestion by savinase, tri-Boc-protected polymyxin E heptapeptide (PMEH(Boc)3) is obtained, which is further conjugated to the separately synthesized building blocks in a convergent synthesis, see FIG. 9 showing scheme 9 for an example of the preparation thereof.

These so-called 2^{nd} generation analogues are prepared as indicating above in Scheme 7. Commercially available polymyxin E, is first treated with Boc-anhydride, yielding the polymyxin species with all free amines Boc-protected. This protected species is subjected to enzymatic digestion by the industrial enzyme savinase, yielding the heptameric macrocycle PMEH(Boc)₃. The preparation of PMEH(Boc)₃ is well-known from literature. Subsequent coupling to a building block, preferably a lipidated tripeptide building block or a building block comprising two amino acids that are connected to a lipid tail with an alpha-amine, followed by global deprotection and purification provides the 2^{nd} generation analogues.

The lipidated tripeptide building blocks are synthesized using solid phase peptide synthesis, as set out in FIG. 9/Scheme 9, showing a representative synthesis of trimeric peptide building blocks used in the preparation of exemplary second generation polymyxin analogues. CTC resin is substituted with the desired amino acid, conjugated via its carboxylic acid. Standard solid phase peptide synthesis (SPPS) procedures yield the desired peptide on resin. This allows for convenient variation of the P3 amino acid as well as optional introduction of a disulfide linked lipid tail. In this set of lipidated tripeptides the N-terminal cysteine is D-Cys. The lipids or disulfide lipids used in the synthesis of the 2^{nd} generation analogues can be selected from the compounds shown above for the 1^{st} generation compounds.

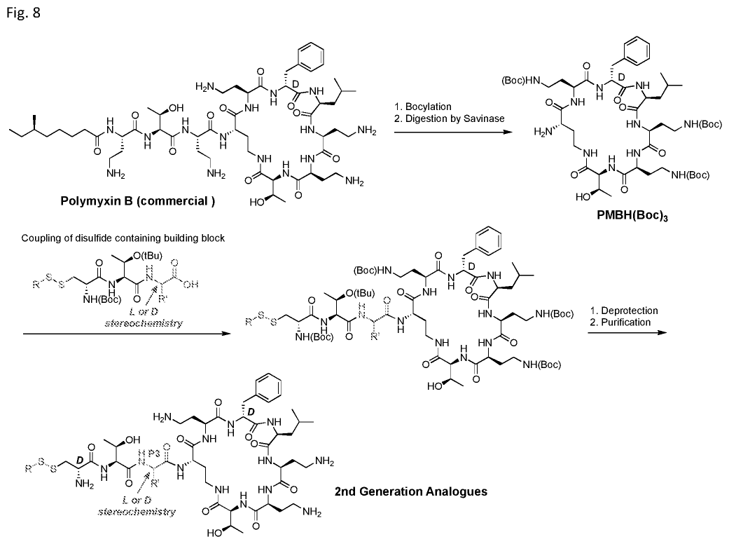

Scheme 8 shows a specific synthesis route to disulfide linked polymyxins including P3 variations. FIG. 8 shows Scheme 8, i.e. the synthesis of disulfide containing polymyxin variants, bearing a non-standard residue at the P3 position as indicated. Compounds are prepared starting from commercially available polymyxin B. After Bocylation and enzymatic digestion by savinase, tri-Boc-protected polymyxin B heptapeptide (PMBH(Boc)3) is obtained, which is further conjugated to the separately synthesized building blocks in a convergent synthesis, see FIG. 9 showing scheme 9 for the preparation thereof.

These so-called 2^{nd} generation analogues were prepared as indicating above in Scheme 2. Commercially available polymyxin B, is first treated with Boc-anhydride, yielding the polymyxin species with all free amines Boc-protected. This protected species is subjected to enzymatic digestion by the industrial enzyme savinase, yielding the heptameric macrocycle PMBH(Boc)₃. The preparation of PMBH(Boc)₃ is well-known from literature. Subsequent coupling to the required lipidated tripeptide building blocks followed by global deprotection and purification provided the 2^{nd} generation analogues.

The required lipidated tripeptide building blocks were synthesized using solid phase peptide synthesis, as set out in FIG. 9/Scheme 9, showing a representative synthesis of trimeric peptide building blocks used in the preparation of the second generation polymyxin analogues. CTC resin was substituted with the desired amino acid, conjugated via its carboxylic acid. Standard solid phase peptide synthesis (SPPS) procedures yielded the desired peptide on resin. This allowed for convenient variation of the P3 amino acid as well as introduction of the desired disulfide linked lipid tail. In this set of lipidated tripeptides the N-terminal cysteine is D-Cys. The disulfide lipids used in the synthesis of the 2^{nd} generation analogues were selected from the compounds shown above for the 1^{st} generation compounds.

The 2^{nd} generation compounds comprise compounds according to Structure BXX:

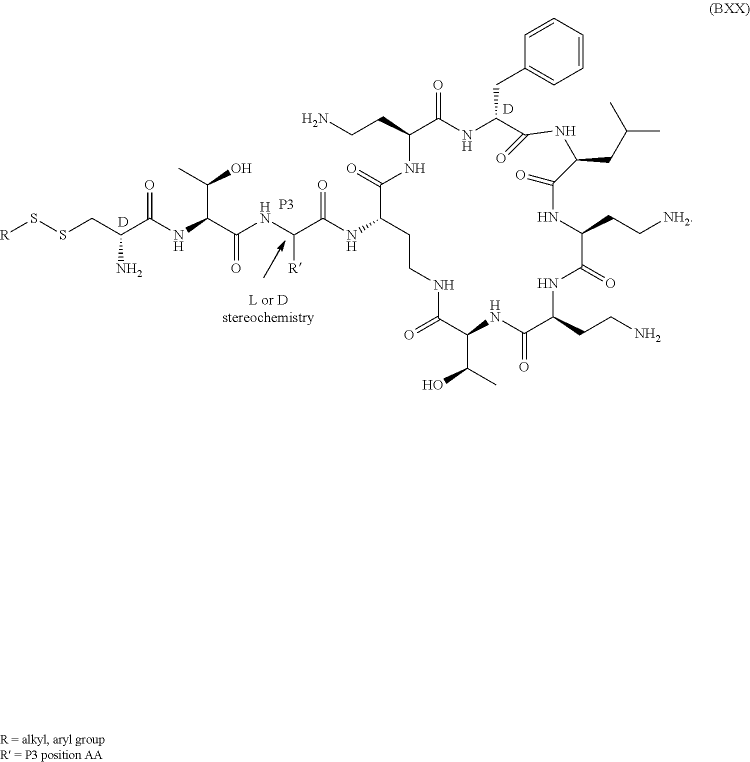

These so-called 2nd generation analogues were prepared as indicating above in FIG. 8. Commercially available polymyxin B, is first treated with Boc-anhydride, yielding the polymyxin species with all free amines Boc-protected. This protected species is subjected to enzymatic digestion by the industrial enzyme savinase, yielding the heptameric macrocycle PMBH(Boc)₃. The preparation of PMBH(Boc)₃ is well-known from literature. Subsequent coupling to the required lipidated tripeptide building blocks followed by global deprotection and purification provided the 2^{nd} generation analogues.

The required lipidated tripeptide building blocks were synthesized using solid phase peptide synthesis (Scheme 9).

Yet further, a set of analogues was prepared, termed 3^{rd} generation compounds, containing a non-amino-acid based linker connecting the nonapeptide and the acyl tail. Those analogues have in common the following linker structure:

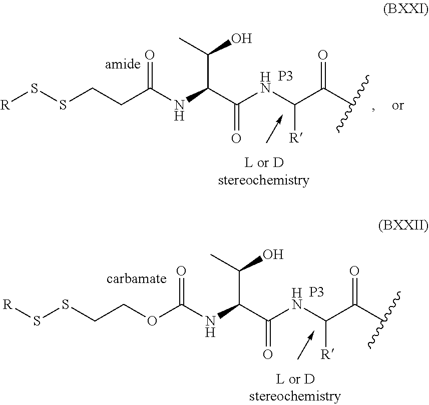

Compounds were prepared via either PMBN(Boc)₄ according to Scheme 2 or 4, or PMBH(Boc)₃ according to Scheme 6 or 8 in case substitution of the amino acid at P3 was desired. Building blocks were the respective carboxylic acids (for amide formation) (structure BXXIII) or chloroformates (for carbamate formation) (structure BXXIV). Preferably, preparation via PMBH(Boc)₃ according to Scheme 6 or 8 comprises the building block having a carboxylic acid group or a hydroxyl group, preferably carboxylic acid group, prior to coupling, at the point of attachment of the building block to the protected and enzymatically digested polymyxin (structure BXXV or BXXVI).

Structures BXXIII, BXXIV, BXXV, and BXXVI are:

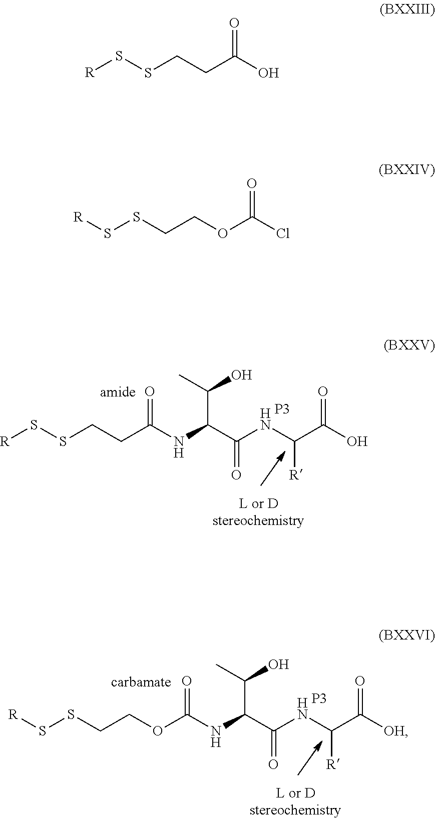

wherein R represents an optionally substituted alkyl moiety, an optionally substituted cycloalkyl moiety; or an optionally substituted aryl moiety, preferably wherein R is an optionally substituted alkyl moiety or an optionally substituted aryl moiety; and

wherein R′ represents hydrogen, hydroxymethyl, aminoethyl, aminomethyl, or 5-(2-amino)pentanoic acid.

Similarly, these 3^{rd} generation compounds can also be prepared by starting with any polymyxin species according to Scheme 1 or according to Scheme 5 in case substitution of the amino acid at P3 is desired. Building blocks can be the respective carboxylic acids (for amide formation) (structure BXXIII) or chloroformates (for carbamate formation) (structure BXXIV). Preferably, preparation to Scheme 5 comprises the building block having a carboxylic acid group or a hydroxyl group, preferably carboxylic acid group, prior to coupling, at the point of attachment of the building block to the protected and enzymatically digested polymyxin (structure BXXV or BXXVI).

Moreover, these 3^{rd} generation compounds can also be prepared via either PMEN(Boc)₄ according to Scheme 3, or PMEH(Boc)₃ according to Scheme 7 in case substitution of the amino acid at P3 is desired. Building blocks can be the respective carboxylic acids (for amide formation) (structure BXXIII) or chloroformates (for carbamate formation) (structure BXXIV). Preferably, preparation via PMEH(Boc)₃ according to Scheme 7 comprises the building block having a hydroxyl group, prior to coupling, at the point of attachment of the building block to the protected and enzymatically digested polymyxin (structure BXXV or BXXVI).

Yet further, Polymyxin analogues containing variation at positions R^{A}—R^{F} are well known in the literature. In addition, stereochemical variants have also be reported. Such polymyxin analogues are accessible by synthetic means accordingly described in the prior art, see for example ACS Cent Sci. 2021, 7, 126-134. DOI: 10.1021/acscentsci.0c01135; and Nature 2022, 601, 606-611. DOI: 10.1038/s41586-021-04264-x.

### EXAMPLES

### Synthesis of Compounds According to the Invention

Compounds were prepared according to scheme 2, 3, 4, 6, 7, or 8 above, preparing different sets of compounds with different substitution patterns. All polymyxin analogues prepared were tested for their anti-bacterial activity on relevant Gram-negative strains (Table B). Polymyxin B and PMBN were taken along as references. In addition, the toxicity on renal Proximal Tubular Epithelial Cells (PTECs) was evaluated.

### Results

Compounds according to the following group of compounds are termed 1st generation compounds herein below, and have the general structure (BXIX)

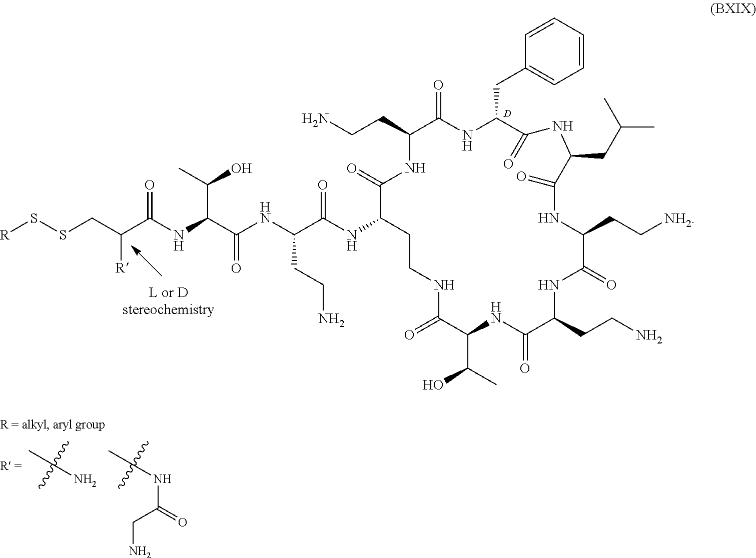

For clarification, as disclosed above, 1^{st} generation compounds do not necessarily contain a disulfide bond nor have to exclusively be based on the polymyxin B structure.

Tables A, A′ and A″ show the MIC values [ug/mL] and relative toxicity values for 1^{st} generation disulfide containing polymyxins. Abbreviations used herein are PMBN: polymyxin B nonapeptide; PTEC: proximal tubular epithelial cell.

TABLE A

| | | | | | | | PTEC |
|---|---|---|---|---|---|---|---|
| | | | | | | | Toxicity |
| | N-terminal | | E. | K. | A. | P. | (relative |
| | Cysteine | | Coli | Pneumoniae | Baumanni | Aeruginosa | to |
| | stereo- | R′ | (ATCC | (ATCC | (ATCC | (ATCC | polymyxin |
| Acyl tail | chemistry | group | 25922) | 13883) | 19606) | 27853) | B) |
| Polymyxin B | | | | | | | |
| — | — | — | 1 | 0.25 | 0.25 | 1 | 1 |
| PMBN | | | | | | | |
| — | — | — | >32 | >32 | >32 | >32 | >10 |
| 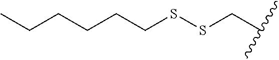 [SMILES: *CSSCCCCCC] | L | 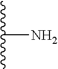 [SMILES: *N] | 8 | 4 | >32 | 2 | 5.3 |
| 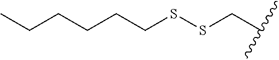 [SMILES: *CSSCCCCCC] | D | 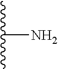 [SMILES: *N] | 2 | 0.5 | 1 | 2 | 3.0 |
| 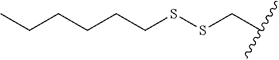 [SMILES: *CSSCCCCCC] | L | 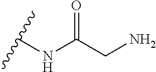 [SMILES: *NC(=O)CN] | 4 | 1 | 2 | 2 | — |
| 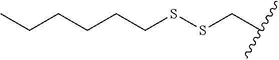 [SMILES: *CSSCCCCCC] | D | 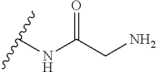 [SMILES: *NC(=O)CN] | 2 | 1 | 1 | 2 | 4.4 |
| 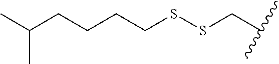 [SMILES: *CSSCCCCC(C)C] | L | 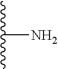 [SMILES: *N] | 2 | 1 | 4 | 2 | — |
| 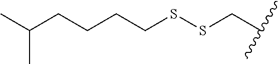 [SMILES: *CSSCCCCC(C)C] | D | 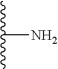 [SMILES: *N] | 4 | 2 | 2 | 16 | 3.3 |
| 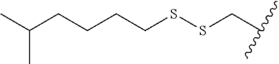 [SMILES: *CSSCCCCC(C)C] | L | 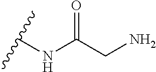 [SMILES: *NC(=O)CN] | 2 | 1 | 1 | 2 | 8.0 |
| 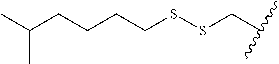 [SMILES: *CSSCCCCC(C)C] | D | 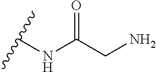 [SMILES: *NC(=O)CN] | 2 | 1 | 1 | 2 | 1.5 |
| 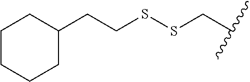 [SMILES: *CSSCCC1CCCCC1] | L | 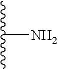 [SMILES: *N] | 8 | 4 | 32 | 4 | — |
| 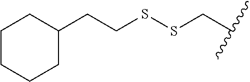 [SMILES: *CSSCCC1CCCCC1] | D | 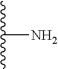 [SMILES: *N] | 1 | 1 | 1 | 2 | 1.2 |
| 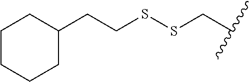 [SMILES: *CSSCCC1CCCCC1] | L | 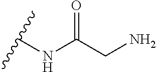 [SMILES: *NC(=O)CN] | 1 | 1 | 1 | 1 | 9.5 |
| 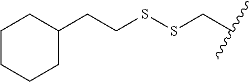 [SMILES: *CSSCCC1CCCCC1] | D | 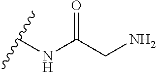 [SMILES: *NC(=O)CN] | 2 | 1 | 1 | 1 | 1.4 |
| 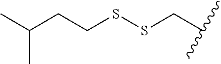 [SMILES: *CSSCCC(C)C] | L | 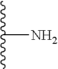 [SMILES: *N] | 16 | 8 | >32 | 2 | 9.8 |
| 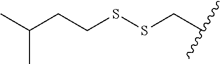 [SMILES: *CSSCCC(C)C] | D | 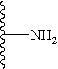 [SMILES: *N] | 1 | 0.5 | 1 | 1 | 4.7 |
| 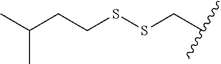 [SMILES: *CSSCCC(C)C] | L | 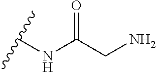 [SMILES: *NC(=O)CN] | 4 | 2 | 4 | 2 | 3.8 |
| 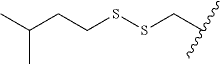 [SMILES: *CSSCCC(C)C] | D | 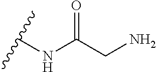 [SMILES: *NC(=O)CN] | 2 | 0.5 | 1 | 1 | 2.0 |

TABLE A′

| | | | | | | | PTEC |
|---|---|---|---|---|---|---|---|
| | N- | | | | A. | P. | Toxicity |
| | terminal | | E. | K. | Bauman- | Aerugi- | (relative |
| | Cysteine | | Coli | Pneumoniae | ni | nosa | to |
| | stereo- | R′ | (ATCC | (ATCC | (ATCC | (ATCC | polymyxin |
| Acyl tail | chemistry | group | 25922) | 13883) | 19606) | 27853) | B) |
| 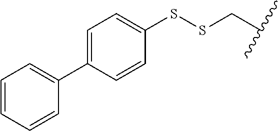 [SMILES: *CSSc1ccc(-c2ccccc2)cc1] | L | 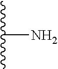 [SMILES: *N] | 4 | 4 | 32 | 8 | — |
| 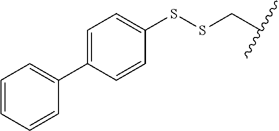 [SMILES: *CSSc1ccc(-c2ccccc2)cc1] | D | 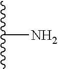 [SMILES: *N] | 8 | 2 | 8 | 8 | — |
| 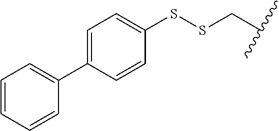 [SMILES: *CSSc1ccc(-c2ccccc2)cc1] | L | 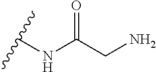 [SMILES: *NC(=O)CN] | 4 | 2 | 16 | 8 | — |
| 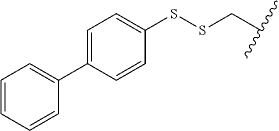 [SMILES: *CSSc1ccc(-c2ccccc2)cc1] | D | 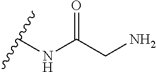 [SMILES: *NC(=O)CN] | 8 | 4 | ND | ND | — |
| 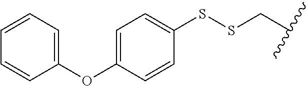 [SMILES: *CSSc1ccc(Oc2ccccc2)cc1] | L | 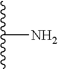 [SMILES: *N] | 4 | 4 | >32 | 4 | — |
| 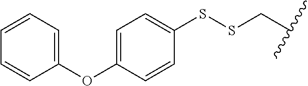 [SMILES: *CSSc1ccc(Oc2ccccc2)cc1] | D | 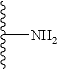 [SMILES: *N] | 4 | 2 | 4 | 4 | — |
| 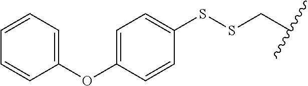 [SMILES: *CSSc1ccc(Oc2ccccc2)cc1] | L | 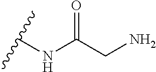 [SMILES: *NC(=O)CN] | 8 | 4 | 32 | 8 | — |
| 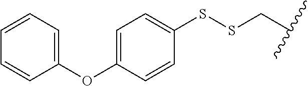 [SMILES: *CSSc1ccc(Oc2ccccc2)cc1] | D | 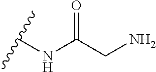 [SMILES: *NC(=O)CN] | 8 | 4 | 8 | 4 | — |
| 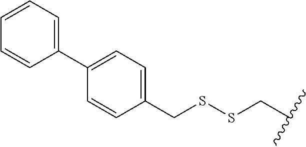 [SMILES: *CSSCc1ccc(-c2ccccc2)cc1] | D | 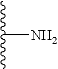 [SMILES: *N] | 4 | 1 | 1 | 2 | 1.7 |
| 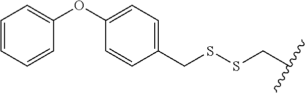 [SMILES: *CSSCc1ccc(Oc2ccccc2)cc1] | D | 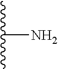 [SMILES: *N] | 4 | 1 | 1 | 4 | 1.8 |
| 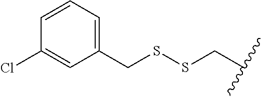 [SMILES: *CSSCc1cccc(Cl)c1] | D | 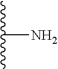 [SMILES: *N] | 1 | 0.5 | 1 | 4 | 1.8 |

TABLE A

| | | | | | | | PTEC |
|---|---|---|---|---|---|---|---|
| | | | | | | | Toxicity |
| | N-terminal | | E. | K. | A. | P. | (relative |
| | Cysteine | | Coli | Pneumoniae | Baumanni | Aeruginosa | to |
| | stereo- | R′ | (ATCC | (ATCC | (ATCC | (ATCC | polymyxin |
| Acyl tail | chemistry | group | 25922) | 13883) | 19606) | 27853) | B) |
| 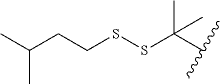 [SMILES: *C(C)(C)SSCCC(C)C] | D | 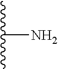 [SMILES: *N] | 2 | 2 | 4 | 2 | — |

The below Example is based on Polymyxin E variant, i.e. identical to the compound immediately above, but
containing D-Leu at position 6 instead of D-Phe (i.e. the phenyl group is replaced by a dimethyl group).

| | | | | | | | |
|---|---|---|---|---|---|---|---|
| 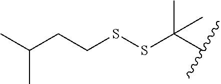 [SMILES: *C(C)(C)SSCCC(C)C] | D | 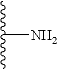 [SMILES: *N] | 2 | 2 | 4 | 2 | — |

The second-generation compounds comprise compounds according to Structure BXX:

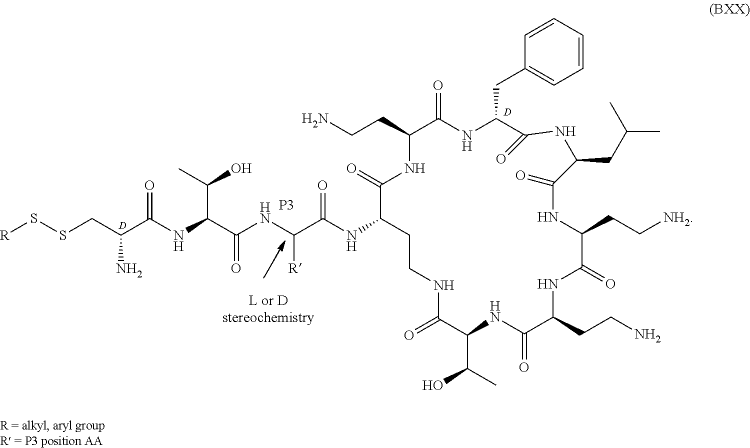

Examples of BXX and two examples of BXX wherein the acyl tail contains a carbon-carbon bond instead of the disulfide bond are set out in Table B. Table B shows MIC values [ug/mL] and relative toxicity values for 2^{nd} generation polymyxins. Abbreviations: Dab: diamino-butyric acid; Dap: diamino-propionic acid; PMBN: polymyxin B nonapeptide; PTEC: proximal tubular epithelial cell.

TABLE B

| | R′ | | | | | |
|---|---|---|---|---|---|---|
| | (P3 | | | | | PTEC |
| | Amino acid | | | | | Toxicity |
| | identity | E. | K. | A. | P. | (relative |
| | and | Coli | Pneumoniae | Baumanni | Aeruginosa | to |
| | stereo- | (ATCC | (ATCC | (ATCC | (ATCC | polymyxin |
| Acyl tail | chemistry) | 25922) | 13883) | 19606) | 27853) | B) |
| Polymyxin B | | | | | | |
| — | Dab (L) | 1 | 0.25 | 0.25 | 1 | 1 |
| PMBN | | | | | | |
| — | Dab (L) | >32 | >32 | >32 | >32 | >10 |
| 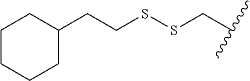 [SMILES: *CSSCCC1CCCCC1] | Dab (L) | 1 | 1 | 0.5 | 2 | 1.2 |
| 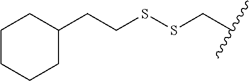 [SMILES: *CSSCCC1CCCCC1] | Dap (L) | 1 | 1 | 0.5 | 2 | 2.9 |
| 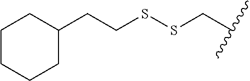 [SMILES: *CSSCCC1CCCCC1] | Ser (D) | 2 | 1 | 0.25 | 4 | 3.3 |
| 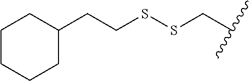 [SMILES: *CSSCCC1CCCCC1] | Gly (-) | 2 | 1 | 0.5 | 2 | 7.4 |
| 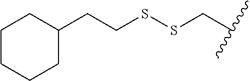 [SMILES: *CSSCCC1CCCCC1] | Dap (D) | 2 | 1 | 0.5 | 2 | 5.4 |
| 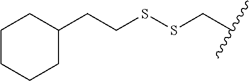 [SMILES: *CSSCCC1CCCCC1] | Dab (D) | 2 | 1 | 0.5 | 2 | 4.8 |
| 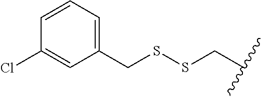 [SMILES: *CSSCc1cccc(Cl)c1] | Dab (L) | 1 | 0.5 | 1 | 4 | 1.8 |
| 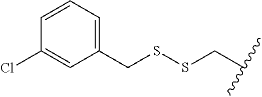 [SMILES: *CSSCc1cccc(Cl)c1] | Dap (L) | 2 | 0.5 | 0.5 | 4 | 4.8 |
| 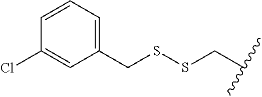 [SMILES: *CSSCc1cccc(Cl)c1] | Ser (D) | 2 | 1 | 0.5 | 4 | 6.7 |
| 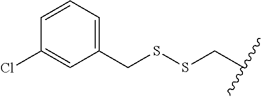 [SMILES: *CSSCc1cccc(Cl)c1] | Gly (-) | 2 | 1 | 1 | 4 | 7.4 |
| 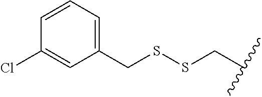 [SMILES: *CSSCc1cccc(Cl)c1] | Dap (D) | 2 | 0.5 | 0.5 | 1 | 5.7 |
| 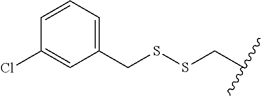 [SMILES: *CSSCc1cccc(Cl)c1] | Dab (D) | 2 | 1 | 0.5 | 2 | 7.0 |
| 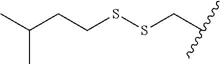 [SMILES: *CSSCCC(C)C] | Dab (L) | 1 | 0.5 | 0.5 | 1 | 9.8 |
| 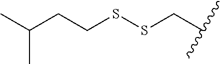 [SMILES: *CSSCCC(C)C] | Dap (L) | 1 | 0.25 | 0.25 | 1 | 20.1 |
| 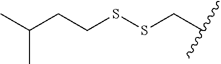 [SMILES: *CSSCCC(C)C] | Ser (D) | 2 | 0.5 | 0.25 | 4 | 21.2 |
| 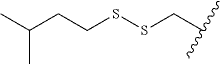 [SMILES: *CSSCCC(C)C] | Gly (-) | 2 | 0.5 | 0.5 | 4 | 26.8 |
| 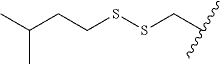 [SMILES: *CSSCCC(C)C] | Dap (D) | 2 | 0.5 | 0.25 | 1 | 9.7 |
| 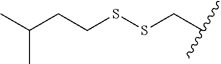 [SMILES: *CSSCCC(C)C] | Dab (D) | 2 | 0.5 | 0.25 | 1 | 7.1 |
| 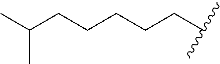 [SMILES: *CCCCCC(C)C] | Dap (L) | 1 | 0.25 | 0.25 | 1 | 7.8 |
| 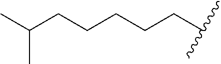 [SMILES: *CCCCCC(C)C] | Dab (L) | 1 | ≤ 0.25 | 0.25 | 1 | 2.0 |
| 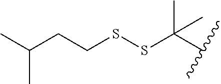 [SMILES: *C(C)(C)SSCCC(C)C] | Dap (L) | 2 | 1 | 1 | 2 | — |

The below Example is based on Polymyxin E variant, i.e. identical to the compound immediately
above, but containing D-Leu at position 6 instead of D-Phe (i.e. the phenyl group is replaced by a
dimethyl group).

| | | | | | | |
|---|---|---|---|---|---|---|
| 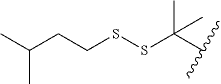 [SMILES: *C(C)(C)SSCCC(C)C] | Dap (L) | 2 | 1 | 2 | 2 | — |

3^{rd} Generation compounds were also subjected to testing for their anti-bacterial activity on relevant Gram-negative strains (Table C). Polymyxin B and PMBN were taken along as references. In addition, the toxicity on renal Proximal Tubular Epithelial Cells (PTECs) was evaluated, see Table C.

The compounds of Table C were prepared via either PMBN(Boc)₄ according to Scheme 4, or PMBH(Boc)₃ according to Scheme 8 in case substitution of the amino acid at P3 was desired. Building blocks were the respective carboxylic acids (for amide formation) or chloroformates (for carbamate formation).

TABLE C
MIC values [ug/mL] and relative toxicity values for 3^{rd} generation disulfide containing polymyxins.

| | | R′ | | | | | |
|---|---|---|---|---|---|---|---|
| | | (P3 | | | | | |
| | | Amino | | | | | PTEC |
| | N-terminal | acid | | | | | Toxicity |
| | amide | identity | E. | K. | A. | P. | (relative |
| | or | and | Coli | Pneumoniae | Baumanni | Aeruginosa | to |
| | carbamate | stereo- | (ATCC | (ATCC | (ATCC | (ATCC | polymyxin |
| Acyl tail | linkage | chemistry) | 25922) | 13883) | 19606) | 27853) | B) |
| Polymyxin B- | — | Dab (L) | 1 | 0.25 | 0.25 | 1 | — |
| PMBN | — | Dab (L) | >32 | >32 | >32 | >32 | >10 |
| 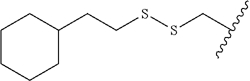 [SMILES: *CSSCCC1CCCCC1] | amide | Dab (L) | 4 | 2 | — | 2 | — |
| 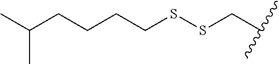 [SMILES: *CSSCCCCC(C)C] | amide | Dab (L) | 4 | 2 | — | 2 | — |
| 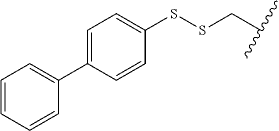 [SMILES: *CSSc1ccc(-c2ccccc2)cc1] | amide | Dab (L) | 8 | 2 | — | 4 | — |
| 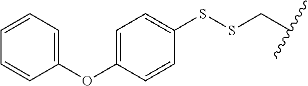 [SMILES: *CSSc1ccc(Oc2ccccc2)cc1] | amide | Dab (L) | 8 | 2 | — | 2 | — |
| 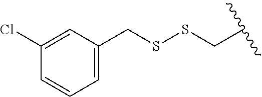 [SMILES: *CSSCc1cccc(Cl)c1] | carbamate | Dab (L) | 4 | 1 | — | 1 | 1.3 |
| 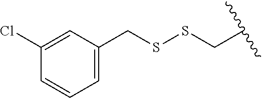 [SMILES: *CSSCc1cccc(Cl)c1] | carbamate | Dap (L) | 8 | 4 | — | 4 | 0.8 |
| 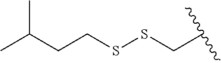 [SMILES: *CSSCCC(C)C] | carbamate | Dab (L) | 8 | 8 | — | 2 | 0.9 |
| 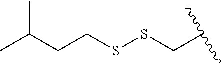 [SMILES: *CSSCCC(C)C] | carbamate | Dap (L) | 4 | 2 | — | 2 | — |
| 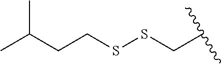 [SMILES: *CSSCCC(C)C] | carbamate | Dap (D) | 4 | 2 | — | 2 | 2.9 |
| 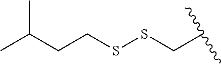 [SMILES: *CSSCCC(C)C] | carbamate | Ser (D) | 4 | 2 | — | 4 | — |

Abbreviations: Dab: diamino-butyric acid; Dap: diamino-propionic acid; PMBN: polymyxin B nonapeptide; PTEC: proximal tubular epithelial cell.

The above examples show that compounds according to the invention may show a superior relationship between toxicity and antibacterial efficacy, thereby permitting to significantly improve the usability of Polymyxine compound in treatment of patients.

1. A compound of formula (I): 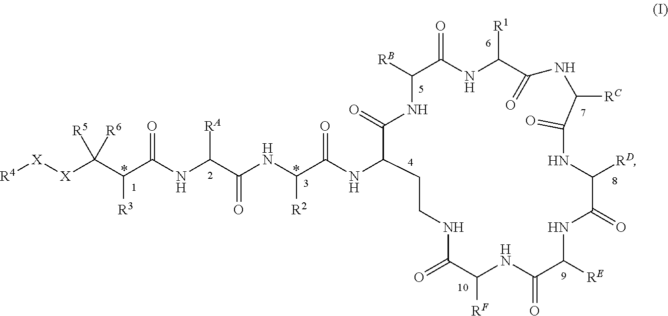
wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R³ represents —NH₂ or —N(H)—COCH₂NH₂;
R⁴ represents an optionally substituted alkyl moiety, an optionally substituted cycloalkyl moiety; or an optionally substituted aryl moiety;
R⁵ and R⁶ each independently represent hydrogen or an optionally substituted alkyl moiety;
each X independently represents C, S, O, or N;
or a pharmaceutically acceptable salt, stereoisomer, solvate, or prodrug thereof.

2. The compound according to claim 1 of formula (Ia): 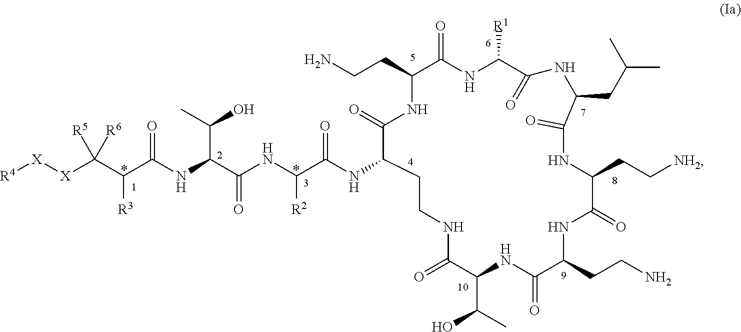
wherein:
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R³ represents —NH₂ or —N(H)—COCH₂NH₂;
R⁴ represents an optionally substituted alkyl moiety, an optionally substituted cycloalkyl moiety; or an optionally substituted aryl moiety;
R⁵ and R⁶ each independently represent hydrogen or an optionally substituted alkyl moiety; or a pharmaceutically acceptable salt, stereoisomer, solvate, or prodrug thereof.

3. The compound according to claim 1 according to formula (II): 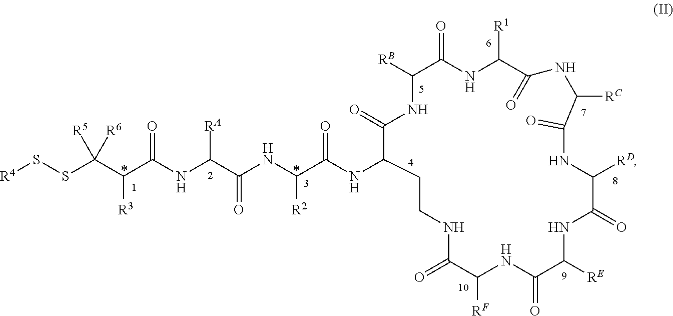
wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃⁺;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R³ represents —NH₂ or —N(H)—COCH₂NH₂;
R⁴ represents an optionally substituted alkyl moiety, an optionally substituted cycloalkyl moiety; or
an optionally substituted aryl moiety; and
R⁵ and R⁶ each independently represent hydrogen or an optionally substituted alkyl moiety; or a pharmaceutically acceptable salt, stereoisomer, solvate, or prodrug thereof.

4. The compound according to claim 1, according to formula (IIa): 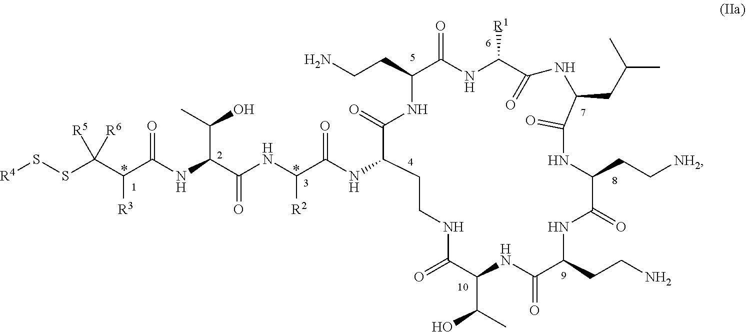
wherein:
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R³ represents —NH₂ or —N(H)—COCH₂NH₂;
R⁴ represents an optionally substituted alkyl moiety, an optionally substituted cycloalkyl moiety; or
an optionally substituted aryl moiety;
R⁵ and R⁶ each independently represent hydrogen or an optionally substituted alkyl moiety; or a pharmaceutically acceptable salt, stereoisomer, solvate, or prodrug thereof.

5. The compound according to claim 1 according to formula (III): 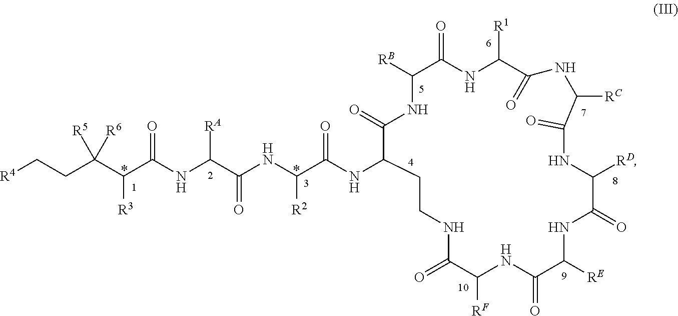
wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃⁺;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R³ represents —NH₂ or —N(H)—COCH₂NH₂;
R⁴ represents an optionally substituted alkyl moiety, an optionally substituted cycloalkyl moiety; or
an optionally substituted aryl moiety; and
R⁵ and R⁶ each independently represent hydrogen or an optionally substituted alkyl moiety; or a pharmaceutically acceptable salt, stereoisomer, solvate, or prodrug thereof.

6. A The compound according to claim 1, according to formula (IIIa): 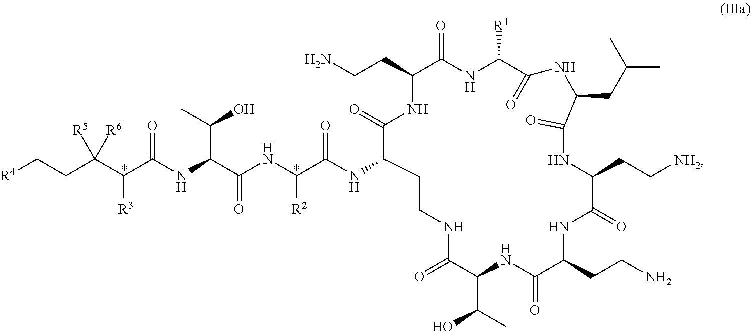
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R³ represents —NH₂ or —N(H)—COCH₂NH₂;
R⁴ represents an optionally substituted alkyl moiety, an optionally substituted cycloalkyl moiety; or
an optionally substituted aryl moiety;
R⁵ and R⁶ each independently represent hydrogen or an optionally substituted alkyl moiety; or a pharmaceutically acceptable salt, stereoisomer, solvate, or prodrug thereof.

7. A The compound according to claim 1 according to formula (IIb): 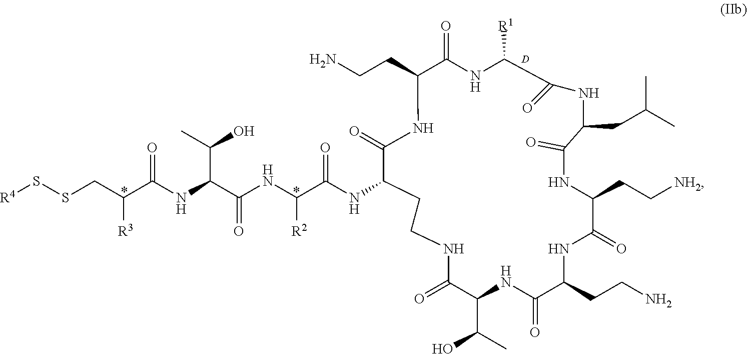
wherein:
R¹ represents an optionally substituted straight or branched chain alkyl, alkenyl, alkinyl or alkylene moiety having up to 20 carbon atoms; optionally substituted with an aryl or heteroaryl moiety;
R² represents hydrogen, hydroxymethyl, 2-aminoethyl, aminomethyl, or 5-(2-amino) pentanoic acid;
R³ represents —NH₂ or —N(H)—COCH₂NH₂; and R⁴ represents an optionally substituted alkyl moiety, an optionally substituted cycloalkyl moiety, or an optionally substituted aryl moiety or an optionally substituted arylalkyl moiety;
or a pharmaceutically acceptable salt, stereoisomer, solvate, or prodrug thereof.

8. A compound of formula (IV): 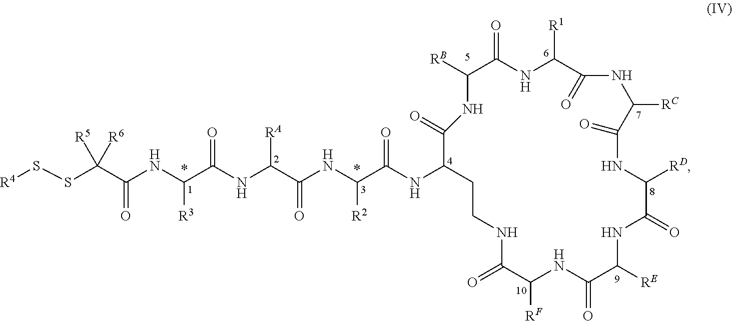
wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃⁺;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R³ represents —NH₂ or —N(H)—COCH₂NH₂;
R⁴ represents an optionally substituted alkyl moiety, an optionally substituted cycloalkyl moiety; or an optionally substituted aryl moiety; and
R⁵ and R⁶ each independently represent hydrogen or an optionally substituted alkyl moiety;
or a pharmaceutically acceptable salt, stereoisomer, solvate, or prodrug thereof.

9. A compound of:
formula (V): 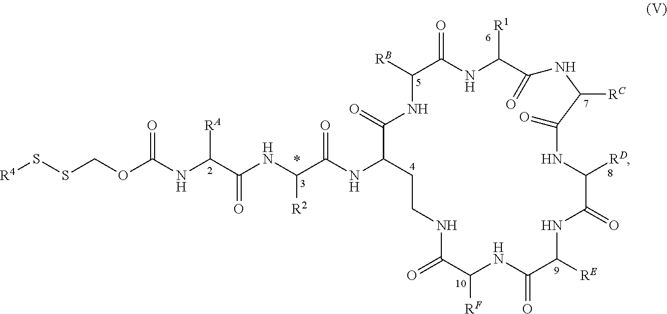
wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃⁺;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R⁴ represents an optionally substituted alkyl moiety, an optionally substituted cycloalkyl moiety; or
an optionally substituted aryl moiety;
or a pharmaceutically acceptable salt, stereoisomer, solvate, or prodrug thereof; or
formula (VI): 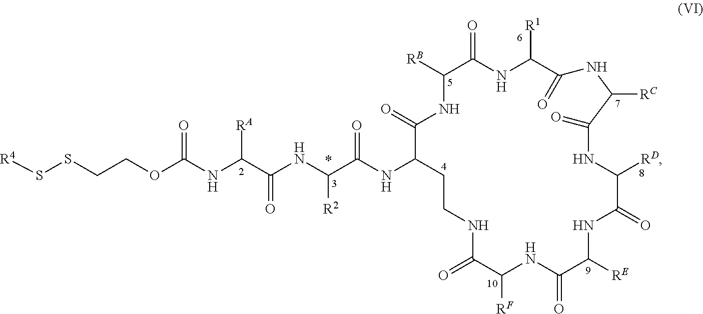
wherein:
R^{A}; R^{B}; R^{C}; R^{D}; R^{E}; and R^{F} each individually represents the side chain of a natural or non-natural α-amino acid in which any functional groups may be protected, preferably wherein R^{A}; R^{B}; R^{C}; R^{D}; R^{E}; and R^{F} each individually represents hydrogen, branched or linear chain C₁-C₄ alkyl, optionally substituted with a hydroxyl, suflhydryl, alkyl thiol ether, carboxyl, in particular a β-carboxyl or a γ-carboxyl, an aromatic or heteroaromatic substituent, in particular benzyl, guanidinium or imidazolium, and/or an amino group, preferably ε-NH₃⁺;
R¹ represents an optionally substituted alkyl moiety, an optionally substituted benzyl moiety;
R² represents hydrogen, hydroxymethyl, aminoethyl, aminomethyl, or 5-(2-amino)pentanoic acid;
R⁴ represents an optionally substituted alkyl moiety, an optionally substituted cycloalkyl moiety; or
an optionally substituted aryl moiety;
or a pharmaceutically acceptable salt, stereoisomer, solvate, or prodrug thereof.

10. (canceled)

11. The compound according to claim 1, wherein R¹ represents a C₁-C₁₀ alkyl moiety, a C₂-C₁₀, preferably monounsaturated, alkenyl moiety or an optionally substituted benzyl moiety.

12. The compound according to claim 1, wherein R⁴ represents a cycloalkyl moiety having of from 4 to 20 carbon atoms, preferably an alicyclic moiety comprising 5, 6 or 7 carbon atoms, wherein the moiety is a monocyclic, bridged or polycyclic ring preferably selected from the groups consisting of cyclopropyl, cyclobutyl, cyclopentyl, cyclohexyl and cycloheptyl; or a pharmaceutically acceptable salt, solvate, or prodrug thereof.

13. The compound according to claim 1, wherein R⁴ represents an optionally substituted arylalkyl moiety, an optionally substituted aryl moiety, a biphenyl moiety.

14. The compound according to claim 1, wherein (*) denominates the stereochemistry at the indicated carbon atom, wherein each may independently be L or D.

15. (canceled)

16. A method of treating a bacterial infection in a subject in need thereof, the method comprising administering the compound of claim 1 to the subject.

17. The method according to claim 16 wherein the bacterial infection is an infection by Gram-negative bacteria, preferably a multi-resistant Gram-negative bacteria strain, more preferably N. meningitidis, E. coli, Salmonella typhi, Bordetella pertussis, Pseudomonas aeruginosa, K. Pneumoniae, A. Baumanni, and/or P. Aeruginosa.

18. An antibacterial composition comprising a compound of claim 1, and a pharmaceutically acceptable carrier.

19. (canceled)

20. A process for the preparation of a compound according to claim 1, comprising:
a) removing the side chain from a polymyxin by enzymatic digestion with an enzyme capable of disrupting the bond between the exocyclic amino acid positioned nearest to the fatty acid tail and the middle exocyclic amino acid to obtain a polymyxin nonapeptide macrocycle comprising a side chain having a free N-terminal amine group;
b) protecting the amino functions of the ring, leaving the N-terminal amine unprotected to obtain a polymyxin nonapeptide macrocycle comprising protected amine groups and a free N-terminal amine group;
c) coupling the free N-terminal amine group with a compound, to obtain a coupled polymyxin nonapeptide macrocycle comprising protected amine groups; and
d) removing the protection groups and isolating the respective obtained compound of formula (I), (Ia), (II), (IIa), (Iib), (III), (IIIa), (IV), (V), or (VI).

21. A process for the preparation of a compound according to claim 1, comprising:
a) protecting the amino functions of a polymyxin to obtain an N-protected polymyxin;
b) removing the complete side chain by enzymatic digestion from the N-protected polymyxin with an enzyme capable of disrupting the bond between the exocyclic amino acid positioned nearest to the cyclic heptapeptide and the cyclic heptapeptide to obtain a N-protected polymyxin heptameric macrocycle with a single free amino group;
a. coupling the free amino group with a lipidated tripeptide or N-terminal amide or carbamate linkage building block, to obtain a coupled polymyxin peptide macrocycle comprising protected amine groups, and
c) removing the protection groups and isolating the respective obtained compound of formula (I), (Ia), (II), (IIa), (IIb), (III), (IIIa), (IV), (V), or (VI).

22.-35. (canceled)

36. A method of treating a bacterial infection in a subject in need thereof, the method comprising administering the composition of claim 18 to the subject.
